(19) United States
(12) Patent Application Publication
(10) Pub. No.: US 2024/0180859 A1
(43) Pub. Date: Jun. 6, 2024

(54) COMPOSITIONS AND METHODS FOR IMMUNE-MEDIATED INFLAMMATORY DISEASES

(71) Applicant: Thetis Pharmaceuticals LLC, Ridgefield, CT (US)

(72) Inventors: Gary Mathias, Ridgefield, CT (US); John Parkinson, Glenside, PA (US); Aaron Mathias, New York, NY (US); Frank C. Sciavolino, Quaker Hill, CT (US)

(73) Assignee: Thetis Pharmaceuticals LLC, Ridgefield, CT (US)

(21) Appl. No.: 18/493,910

(22) Filed: Oct. 25, 2023

Related U.S. Application Data
(60) Provisional application No. 63/380,981, filed on Oct. 26, 2022.

(51) Int. Cl. A61K 31/198; A61K 31/315; A61K 45/06; A61P 35/00

(52) U.S. Cl. CPC A61K 31/198; A61K 31/315; A61K 45/06; A61P 35/00

(57) ABSTRACT

The present invention relates to compositions comprising resolvins and their use in methods of treating immune-mediated inflammatory disease.

### CROSS REFERENCE TO RELATED APPLICATIONS

This is a non-provisional of pending U.S. provisional patent application Ser. No. 63/380,981, filed Oct. 26, 2022, the entirety of which application is incorporated by reference herein.

### FIELD OF THE INVENTION

The present invention relates to compositions comprising resolvins and salts thereof, and their use in therapy, particularly in the treatment of immune-mediated inflammatory diseases.

### BACKGROUND

The inflammatory response in animals has two phases: initiation and resolution. At the cellular level, initiation is characterized by edema and the accumulation of immune cells such as neutrophils, monocytes, and macrophages. The initiation phase of the inflammatory response has long been recognized as an active process driven by metabolites of arachidonic acid such as the prostaglandins PGE₂ and PGD₂, which are chemo-attractants for eosinophils, neutrophils and monocytes, and the leukotrienes, especially LTB₄ which elicit adhesion, chemotaxis, and aggregation of leukocytes. In order for the inflamed tissue to return to a healthy state, the excess inflammatory cells, cellular debris, and other remnants of the host defense and any invading microorganisms must be cleared. This ‘resolution’ phase of the inflammatory response was for many years believed to be a passive process, the result of the dilution of the chemo-attractants of the initiation phase.

Today, resolution of inflammation is recognized as an active process, driven by various molecules that counteract the pro-inflammatory effects of prostaglandins and leukotrienes. For example, resolvins are autacoids produced locally at the site of inflammation that promote the resolution of inflammation by recruiting non-inflammatory monocytes, which differentiate into macrophages to remove excess neutrophils and cellular debris. Resolvins are part of a class of ‘specialized pro-resolving mediators’ (“SPMs”) of inflammation that also include lipoxins, protectins, and maresins. Serhan et al., “Lipid Mediators of Inflammation”, Cold Spring Harb Perspect Biol 2015; 7:a016311.

Unresolved inflammation is widely recognized as a unifying component in many immune-mediated inflammatory diseases, including cancer. Serhan, C. N., Nature 2014 510:92-101; Coussens et al. Nature 2002; 420, 860-867, Grivennikov et al. Cell. 2010; 140(6):883-99, Todoric et al. Cancer Prev Res. 2016; 9(12):895-905; Fishbein et al. Pharmacol Ther. 2021; 218:107670. With respect to cancer, experimental studies demonstrate that inflammation can stimulate tumor initiation, growth, and metastasis, and observational clinical studies suggest that systemic inflammation in cancer patients is associated with worse survival outcomes. Thomsen et al., Oncotarget. 2016 Nov. 15; 7(46):75013-75022k; Babic et al., Clin Transl Gastroenterol. 2018 Apr. 25; 9(4):145.

Using preclinical animal models, Sulciner describes the use of continuous intraperitoneal (IP) infusion of three well-characterized SPMs including resolvin E1 (RvE1), resolvin D1 (RvD1), and resolvin D2 (RvD2) to treat solid tumor cancers in preclinical models of lymphoma, lung cancer, breast cancer, and pancreatic cancer. The use of continuous infusion enabled much lower doses to be used compared with previous studies that relied on IP injection. Both dosing regimens were premised on the need to maintain systemic exposure of the resolvins due to their rapid metabolic inactivation and consequently short biological half-life. Arita et al. J Biol. Chem. 281,32 (2006): 22847-54; Xu et al, Nat. Med 2010; Yoo et al. Curr. Neuropharmacol. (2013); Obsorov et al. J Neuro. Neurophy. 2017; Valdes et al. Sci. Reports (2017); Hesselink. Pain and therapy vol. 6,2 (2017); Lavy et al., Front. Immunol., 12, 1 (2021).

Arita determined that oxidoreductase enzymes conserved in all mammalian species catalyze the conversion of RvE1 into 18-oxo-RvE1, which was found to be “devoid of activity” in zymosan-induced peritonitis, whereas an equimolar dose of “RvE1 potently reduced polymorphonuclear leukocyte (PMN) recruitment”. Arita also notes that resolvins undergo metabolic inactivation that is similar to other “eicosanoids such as prostaglandins, leukotrienes, and lipoxins, which are generated from stored precursors, exert their bioactions at specific receptors, and are rapidly inactivated via local enzymatic catabolism in target tissues.” Investigating the analgesic effects of intrathecal administration of natural RvE1 compared to a stable RvE1 analog, Xu demonstrated that of the effects of the analog “designed to resist rapid local metabolic inactivation of RvE1” lasted 6 hours or 3 times longer than natural RvE1.

Yoo reports that resolvins are “highly unstable lipids compared to their precursors such as DHA and EPA” and that “the natural forms are likely prone to degradation or metabolism”. Valdes notes that “resolvins are rapidly degraded in vivo resulting in a short biological half-life”. In a report on the therapeutic use of lipid autacoids including resolvins, Hesselink notes that such compounds have “great promise” but require significant formulation development because “most compounds have a short half-life”. This obstacle to therapeutic use of natural resolvins and other SPMs for treatment of cancer is also highlighted by Lavy, noting that “native SPMs” have the disadvantage of a “short half-life.”

Thus, despite their potential as anti-cancer agents reported in Sulciner and others, clinical development of resolvins has been hindered, in part by the insufficient bioavailability and rapid clearance of these molecules. One way to overcome the “restricted bioavailability and rapid clearance” of specialized proresolving mediators, such as resolvins, was provided by Trilleaud et al., Sci. Adv. 2021. Trilleaud describes the use of a monoclonal antibody developed to activate ChemR23, thereby eliminating the need to administer its natural ligand, RvE1. The rationale for using the extended plasma half-life ChemR23 agonist antibody was to overcome the “restricted bioavailability and rapid clearance” of SPMs, such as resolvins. Another approach to overcome the short half-life of resolvins that is used in many preclinical studies of cancer therapy was to administer the resolvin by daily intraperitoneal (IP) injection or using a continuous infusion regimen, such as an IP osmotic pump. Ye et al. (2018); Sun et al.; Sulciner et al. (2018); US 20200330551.

Others have reported the use of resolvins as a neo-adjuvant therapy. Debulking and curative treatment for locoregional disease, such as surgery or radiation therapy, can induce tumor-dormancy escape and subsequent metastatic outgrowth by impairing tumor-specific immunity through inflammation-mediated growth signals and deficient resolution of inflammation. Therefore, in the treatment of cancer with surgical resection or radiation therapy, there is an opportunity in the perioperative period to lower the risk of metastatic growth and relapse via the use of neo-adjuvant therapy to reduce dormancy escape of cancer cells and counter the pro-tumor inflammatory effects of the debulking or curative treatment of the primary tumor. Addressing this opportunity, Panigrahy determined that perioperative use of D-series resolvins administered continuously via intraperitoneal pump beginning two hours before surgery markedly improves survival following tumor resection in the metastatic LLC resection model. Panigrahy D et al. J Clin Invest. 2019; 129(7):2964-2979.

Others have reported the use of resolvins as preventive agents. For example, Kuang et al. (2016) describes chemoprevention with resolvins to alleviate the progression of hepatitis toward liver cancer in a long-term concanavalin A-induced murine model. Notably, Kuang observed that administration of either RvD1 or RvE1 in this model system markedly downregulated levels of CD4+ and CD8+ cells in the liver, which is a marker of poor prognosis and response to treatment in cancer. Ostroumov et al. Cell Mol Life Sci (2018). US 20200330551 describes methods for preventing or treating cancer comprising overexpressing of the GPCR receptor of RvE1 known as ChemR23, which may be combined with administering RvE1 where the RvE1 was administered by IP injection prior to or at the time of tumor cell implantation, which does not mimic the clinical setting in which patients present with established tumors or metastatic disease.

There is a need for new compositions and methods to exploit the therapeutic potential of resolvins in immune-mediated inflammatory diseases. The present invention addresses these needs.

### BRIEF SUMMARY

The present invention relates to compositions comprising a resolvin, or a salt thereof, for use in the treatment of immune-mediated inflammatory diseases (IMIDs). Resolvins are quickly cleared following intravenous or subcutaneous administration, exhibiting a serum half-life of less than one hour. In order to increase bioavailability, prior studies of resolvin E1 for treating IMIDs utilized a continuous dosing regimen such as intraperitoneal (IP) administration by a mini-osmotic pump, or daily IP injection. The compositions and methods described here are based, in part, on unexpected findings of efficacy for resolvins and salts of resolvins administered by less than daily (LTD) dosing, and in certain embodiments dosing every six days (Q6D) or seven days (Q7D).

Accordingly, the disclosure provides methods for treating IMIDs in a subject in need of such treatment comprising administering to the subject a pharmaceutical composition comprising a resolvin, or a salt thereof. In embodiments, the resolvin or its salt is administered by a non-continuous route, such as by subcutaneous injection or oral dosing. In embodiments, the resolvin or its salt is administered in a dosing regimen of once daily or less than daily (LTD) dosing, for example once every two days (Q2D), or once every three days (Q3D), or once every six days (Q6D), or once every seven days (Q7D). In embodiments, the resolvin or its salt is administered in a dosing regimen of once every three days (Q3D), or once every six days (Q6D), or once every seven days (Q7D). In embodiments, the resolvin or its salt is administered in a dosing regimen of once every two weeks (Q14D), once every three weeks (Q21D), or once every four weeks (Q28D).

In embodiments, the compositions for use in the methods described here comprise a salt of a resolvin. In embodiments, the resolvin salt is peptide or mineral amino acid salt form of a resolvin or its aspirin-triggered counterpart described in Formulas I-IV. The resolvin salts described in Formulas I-IV contain two resolvin molecules ionically bound to at least one basic function provided by a scaffold. For example, in compounds of Formulas I and III, the scaffold is peptide-based; in compounds of Formula IV, the scaffold is a divalent metal-amino acid chelate or divalent metal-peptide chelate; and in compounds of Formula II, the scaffold is either a dipeptide or a monovalent metal or non-metal dipeptide.

In embodiments, the disclosure provides methods for treating IMIDs in a subject in need of such treatment, the methods comprising administering to the subject a pharmaceutical composition comprising a resolvin, or a salt thereof, in a therapeutic regimen of less than once daily, either alone as monotherapy or as adjuvant therapy, optionally in combination with at least one additional therapeutic agent as described herein.

In embodiments, the resolvin is a sodium, potassium, or magnesium salt.

In embodiments, the resolvin is a mineral amino acid salt of Formula IV, or an enantiomer, polymorph, solvate, or hydrate thereof:

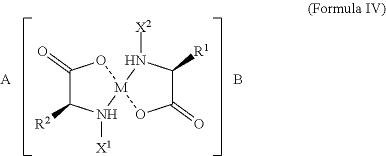

wherein
M is a divalent metal selected from magnesium (Mg²⁺), calcium (Ca²⁺), and zinc (Zn²⁺);
A and B are each independently a resolvin molecule;
A and B may be the same or different;
either A or B, but not both, may be absent;
R¹ and R² are each independently a C₁-C₁₀ alkyl comprising at least one basic function; and
X¹ and X² are each independently H or CO—Z and Z is a peptide comprising 1 to 5 amino acids.

In embodiments, the resolvin is an E or D series resolvin, optionally wherein the E series resolvin is selected from the group consisting of resolvin E1 (RvE1), resolvin E2 (RvE2), resolvin E3 (RvE3), resolvin E4 (RvE4), aspirin-triggered RvE1 (AT-RvE1), AT-RvE2, and AT-RvE3, and further optionally wherein the D series resolvin selected from the group consisting of resolvin D1 (RvD1), resolvin D2 (RvD2), resolvin D3 (RvD3), resolvin D4 (RvD4), resolvin D5 (RvD5), resolvin D6 (RvD6), aspirin-triggered resolvin D1 (AT-RvD1), AT-RvD2, AT-RvD3, AT-RvD4, AT-RvD5, and AT-RvD6. In embodiments, the resolvin is RvE1, RvD1, or RvD2.

In embodiments, A and B are each independently an E series resolvin selected from the group consisting of resolvin E1 (RvE1), resolvin E2 (RvE2), resolvin E3 (RvE3), resolvin E4 (RvE4), aspirin-triggered RvE1 (AT-RvE1), AT-RvE2, and AT-RvE3.

In embodiments, A and B are each independently a D series resolvin selected from the group consisting of resolvin D1 (RvD1), resolvin D2 (RvD2), resolvin D3 (RvD3), resolvin D4 (RvD4), resolvin D5 (RvD5), resolvin D6 (RvD6), protectin D1, protectin DX, aspirin-triggered resolvin D1 (AT-RvD1), AT-RvD2, AT-RvD3, AT-RvD4, AT-RvD5, AT-RvD6, and AT protectin D1

In embodiments, M is selected from magnesium (Mg²⁺) or calcium (Ca²⁺). In embodiments, R¹ and R² are each independently —(CH₂)₃—Y¹, and —(CH₂)₄—Y², and Y¹ and Y² are each selected from a positively charged primary amine, a positively charged secondary amine, a positively charged tertiary amine, and a positively charged guanidine. In embodiments, X¹ and X² are each H.

In embodiments, R¹ and R² are each —(CH₂)₄—Y² and Y² is —NH₃⁺

In embodiments, A and B are the same.

In embodiments, M is magnesium (Mg²⁺), R¹ and R² are each —(CH₂)₄—Y² and Y² is —NH₃⁺, X¹ and X² are each H, and A and B are RvE1, which compound is referred to as bis RvE1 Mg-di-lysinate (Compound 1).

In embodiments, M is magnesium (Mg²⁺), R¹ and R² are each —(CH₂)₄—Y² and Y² is —NH₃⁺, X¹ and X² are each H, and A and B are RvD1, which compound is referred to as bis RvD1 Mg-di-lysinate.

In embodiments, M is magnesium (Mg²⁺), R¹ and R² are each —(CH₂)₄—Y² and Y² is —NH₃⁺, X¹ and X² are each H, and A and B are RvD2, which compound is referred to as bis RvD2 Mg-di-lysinate.

In embodiments of any of the foregoing methods, the immune-mediated inflammatory disease (IMID) is selected from the group consisting of inflammatory bowel disease (IBD), ulcerative colitis, Crohn's disease, proctitis, pouchitis, Crohn's disease of the pouch, eosinophilic colitis, lymphocytic colitis, collagenous colitis, diversion colitis, chemical colitis, ischemic colitis, eosinophilic esophagitis, Behcet's disease, irritable bowel syndrome, Celiac disease, intestinal mucositis, diverticulitis, and short bowel syndrome, optionally wherein the inflammatory disease or disorder is ulcerative colitis, checkpoint inhibitor-induced colitis, Crohn's disease, or pouchitis.

In embodiments, the immune-mediated inflammatory disease is selected from the group consisting of acne, adipose tissue inflammation, allograft rejection, arthritis, bacterial infection, bullous pemphigoid, burn wounds, chelitis, chronic pancreatitis, corneal wound, dermatitis, diabetic wounds, dry eye syndrome, eczema, endometriosis, endotoxin shock, epidermolysis, ankylosing spondylitis, bullosa acquisita, glossitis, heart ischemia, HSV-keratitis, hidradenitis suppurativa, IgA-mediated bullous dermatoses, ischemia reperfusion injury, localized aggressive periodontitis, lupus erythematosus, lyme arthritis, macular edema, oral mucositis, osteoarthritis, periodontitis, peritonitis, pemphigus, postoperative pain, postsurgical cognitive decline, pruritus, psoriasis, psoriatic arthritis, pyoderma gangrenosum, retinopathy, rheumatoid arthritis, scleroderma, Sjogren's syndrome, steroid-induced rosacea, stomatitis, systemic inflammatory response syndrome, temporomandibular joint inflammation, and vascular inflammation.

In embodiments, wherein the immune-mediated inflammatory disease is selected from the group consisting of bacterial pneumonia, tuberculosis, sepsis, and sepsis-induced cardiomyopathy.

In embodiments, wherein the immune-mediated inflammatory disease is selected from the group consisting of type 2 diabetes, insulin resistance, hypertriglyceridemia, mixed dyslipidemia, hypercholesterolemia, fatty liver, hypertriglyceridemia, hypercholesterolemia mixed dyslipidemia, nonalcoholic steatohepatitis (NASH), primary biliary syndrome, and primary schlerosing cholangitis.

In embodiments, the immune-mediated inflammatory disease is selected from the group consisting of Alzheimer's disease, peripheral nerve injury, amyotrophic lateral sclerosis, multiple sclerosis, pain, and fibromyalgia.

In embodiments, the immune-mediated inflammatory disease is selected from the group consisting of asthma, pulmonary inflammation, bronchiolitis obliterans, bronchopulmonary dysplasia, also referred to as chronic lung disease of infancy, cystic fibrosis, allergic airway response, acute lung injury, acute respiratory distress syndrome, lung injury, idiopathic pulmonary fibrosis, bacterial pneumonia, cigarette smoke-induced lung inflammation, and vascular inflammation.

In embodiments, the immune-mediated inflammatory disease is a cancer selected from the group consisting of brain cancer, breast cancer, bladder cancer, colorectal cancer, esophageal cancer, gastric cancer, head and neck cancer, liver cancer, lung cancer, melanoma or other skin cancer, ovarian cancer, pancreatic cancer, prostate cancer, renal cell carcinoma, and sarcoma.

In embodiments, the method comprises administering the pharmaceutical composition less than daily (LTD), optionally every two days (Q2D), every three days (Q3D), every six days (Q6D), every seven days (Q7D), every fourteen days (Q14D), every twenty-one days (Q21D), or every twenty-eight days (Q28D). In embodiments, LTD administration may be at weekly intervals, for example every two weeks (Q14D), every three weeks (Q21D), or every four weeks (Q28D).

In embodiments, the resolvin or its salt is administered in a dosing regimen of once every three days (Q3D), or once every six days (Q6D), or once every seven days (Q7D).

In embodiments, the resolvin is administered orally or parenterally, optionally wherein the parenteral administration is subcutaneous, intraperitoneal, intramuscular, or intravenous. In embodiments, the resolvin is administered by inhalation. In embodiments, the resolvin is administered by a sublingual route.

In embodiments, the method comprises administering the resolvin, or a salt thereof, in combination with one or more additional therapeutic agents, such as chemotherapy, targeted therapy, immunotherapy, hormone therapy, CAR T cell therapy, gene therapy, or microbiome therapy.

The disclosure also provides for the use of a pharmaceutical composition comprising a resolvin, or a salt thereof, in a method for treating immune-mediated inflammatory diseases (IMIDs) in a subject, wherein the composition is adapted for administration less than once daily.

In embodiments of any of the foregoing methods, the subject is a human subject.

Other technical features may be readily apparent to one skilled in the art from the following figures, descriptions, and claims.

### BRIEF DESCRIPTION OF THE FIGURES

FIG. 1A is a line graph showing tumor volume over time (Day 15 vs Day 0) for bis RvE1 Mg-di-lysinate in the MC38 model of colorectal cancer.

FIG. 1B is a scatter plot showing percent change in tumor volume (Day 15 vs Day 0) for bis RvE1 Mg-di-lysinate in the MC38 model of colorectal cancer.

FIG. 2A is a line graph showing tumor volume over time (Day 20 vs Day 0) for bis RvE1 Mg-di-lysinate in the Panc2-H7 model of pancreatic cancer with daily (QD) and weekly (Q7D) dosing.

FIG. 2B is a scatter plot showing percent change in tumor volume (Day 20 vs Day 0) for bis RvE1 Mg-di-lysinate in the Panc2-H7 model of pancreatic cancer with daily (QD) and weekly (Q7D) dosing.

FIG. 3A is a line graph showing plasma levels of RvE1 over time in mice following subcutaneous dosing with bis RvE1 Mg-di-lysinate.

FIG. 3B is a line graph showing plasma levels of RvE1 over time in mice following subcutaneous dosing with RvE1-Na.

FIG. 3C is a line graph showing plasma levels of RvE1 over time in mice following intravenous dosing with RvE1-Na.

FIG. 3D is a line graph showing plasma levels of RvE1 over time in mice following oral dosing with RvE1-Na.

FIG. 3E is a line graph showing lung tissue levels of RvE1 over time in mice following subcutaneous dosing with RvE1-Na.

FIG. 3F is a line graph showing lung tissue levels of RvE1 over time in mice following intravenous dosing with RvE1-Na.

FIG. 3G is a line graph showing lung tissue levels of RvE1 over time in mice following oral dosing with RvE1-Na.

FIG. 4 is a heat map showing activation of adaptive immune response in T cells and other immune cells in pancreatic tumors following treatment with bis RvE1 Mg-di-lysinate.

### DETAILED DESCRIPTION

The present invention relates to compositions comprising a resolvin, or a salt thereof, for use in the treatment of immune-mediated inflammatory diseases (IMIDs). Despite their potential anti-inflammatory and pro-resolving effects, translation of resolvins into the clinic has been hampered, in part, by expectations that their rapid clearance would restrict their bioavailability. Resolvins are quickly cleared following intravenous or subcutaneous administration, exhibiting a serum half-life of less than one hour due to their rapid metabolic inactivation, which was first elucidated by Arita et al., J. Biol. Chem. 281,32 (2006): 22847-54. In order to increase bioavailability, prior studies showing therapeutic efficacy utilized a continuous or daily dosing regimen such as administration by a mini-osmotic pump or a daily regimen using IP injection, which generally has a rate of absorption that is one half to one-fourth as rapid as IV administration. Al Shoyaib et al. Pharmaceut.l Res. 37,1 12. 23 Dec. 2019. The compositions and methods described here are based, in part, on unexpected findings of efficacy for resolvins and salts of resolvins administered once every six or seven days (Q6D or Q7D).

Continuous or daily regimens are more difficult to translate into the clinic due to reduced patient adherence to such regimens. Daily dosing, whether by the oral, subcutaneous or other routes of administration, places a significant burden on patients, and has been shown to have lower adherence compared to less frequent administration, such as weekly dosing. Weeda et al., Int J Clin Pract. 2021 September; 75(9):e14060; Hohneker et al., J Oncol Pract. 2011; 7:65-67. Despite the extensive use of weekly (Q7D) dosing for biologics and immunomodulators to treat inflammatory diseases, this approach has not been reported or deemed feasible with resolvins.

The present invention advantageously provides methods and compositions for the treatment of immune-mediated inflammatory diseases (IMIDs) using resolvins administered in therapeutic regimens at less than daily (LTD) dosing and by a non-continuous route of administration, such as subcutaneous, intravenous, oral, inhalable or sublingual administration. In embodiments, the resolvin is administered LTD, optionally every two days (Q2D), every three days (Q3D), every six days (Q6D), every seven days (Q7D) or even less frequently such as once every two weeks (Q14D) or once every four weeks (Q28D). The compositions and methods described here are based, in part, on unexpected findings of efficacy for resolvins and salts of resolvins administered by a non-continuous route, and in certain embodiments at less than daily (LTD) dosing. The invention further provides methods relating to combination therapy with resolvins, which are described in the following sections.

In embodiments of the compositions and methods described here, the resolvin is in the form of a simple salt, such as a sodium, potassium, or magnesium salt. In embodiments, the resolvin is in the form of a salt described by a compound of Formula I-IV. In embodiments, the disclosure provides a pharmaceutical composition comprising a compound of Formula IV which is a magnesium L-lysinate bis resolvin E1, a magnesium L-lysinate bis resolvin D1, or a magnesium L-lysinate bis resolvin D2, for use in a method of treating immune-mediated inflammatory diseases (IMIDs) as described herein.

In embodiments, the methods of treating immune-mediated inflammatory diseases (IMIDs) described here comprise administering a pharmaceutical composition comprising a compound of Formula IV to a human subject in need of treatment. In embodiments, the compound of Formula IV is a mono or bis resolvin magnesium, calcium, or zinc di-lysinate (M-lys-lys, or M-di-lysinate) compound selected from the group consisting of RvE1 M-lys-lys, RvE2 M-lys-lys, RvE3 M-lys-lys, RvE4 M-lys-lys, AT-RvE1 M-lys-lys, AT-RvE2 M-lys-lys, and AT-RvE3 M-lys-lys. In embodiments, the compound of Formula IV is a mono or bis RvE1 Mg-lys-lys or a mono or bis AT-RvE1 Mg-lys-lys.

In embodiments, the methods of treating immune-mediated inflammatory diseases (IMIDs) described here comprise administering a pharmaceutical composition comprising a lysyl-lysine dipeptide of Formula I where A and B are the same and are selected from an E or D series resolvin. In embodiments, the E series resolvin is selected from RvE1, RvE2, RvE3, RvE4, AT-RvE1, AT-RvE2, and AT-RvE3. In embodiments, the D series resolvin is selected from RvD1, RvD2, RvD3, RvD4, RvD5, RvD6, AT-RvD1, AT-RvD2, AT-RvD3, AT-RvD4, AT-RvD5, and AT-RvD6. In embodiments, the compound is a bis salt of the resolvin. In embodiments, the compound is selected from the group consisting of mono or bis RvE1 lysyl lysine, mono or bis AT-RvE1 lysyl lysine, mono or bis RvE2 lysyl lysine, mono or bis AT-RvE2 lysyl lysine, mono or bis RvE3 lysyl lysine, mono or bis AT-RvE3 lysyl lysine, and mono or bis RvE4 lysyl lysine.

In embodiments the methods of treating immune-mediated inflammatory diseases (IMIDs) described here comprise administering a pharmaceutical composition comprising a linear lysyl-lysine dipeptide of Formula III where A and B are the same and are selected from an E or D series resolvin. In embodiments, the E series resolvin is selected from RvE1, RvE2, RvE3, RvE4, AT-RvE1, AT-RvE2, and AT-RvE3. In embodiments, the D series resolvin is selected from RvD1, RvD2, RvD3, RvD4, RvD5, RvD6, AT-RvD1, AT-RvD2, AT-RvD3, AT-RvD4, AT-RvD5, and AT-RvD6. In embodiments, the compound is a bis salt of the resolvin. In embodiments, the compound is selected from the group consisting of mono or bis RvE1 linear lysyl lysine, mono or bis AT-RvE1 linear lysyl lysine, mono or bis RvE2 linear lysyl lysine, mono or bis AT-RvE2 linear lysyl lysine, mono or bis RvE3 linear lysyl lysine, and mono or bis AT-RvE3 linear lysyl lysine.

In embodiments, the methods of treating immune-mediated inflammatory diseases (IMIDs) described here comprise administering a pharmaceutical composition comprising a compound selected from a magnesium, calcium, or zinc di-lysinate of Formula IV where A and B are the same and are selected from an E or D series resolvin. In embodiments, the E series resolvin is selected from RvE1, RvE2, RvE3, RvE4, AT-RvE1, AT-RvE2, and AT-RvE3. In embodiments, the D series resolvin is selected from RvD1, RvD2, RvD3, RvD4, RvD5, RvD6, AT-RvD1, AT-RvD2, AT-RvD3, AT-RvD4, AT-RvD5, and AT-RvD6. In embodiments, the compound is a bis salt of the resolvin. In embodiments, the compound is selected from the group consisting of mono or bis RvE1 Mg-di-lysinate, mono or bis AT-RvE1 Mg-di-lysinate, mono or bis RvE2 Mg-di-lysinate, mono or bis AT-RvE2 Mg-di-lysinate, mono or bis RvE3 Mg-di-lysinate, mono or bis AT-RvE3 Mg-di-lysinate, and mono or bis RvE4 Mg-di-lysinate.

In the context of any of the methods of the present invention, the subject may be a human or a non-human mammal. The non-human mammal may be, for example, a non-human primate, a dog, cat, a rodent (e.g., a mouse, a rat, a rabbit), a horse, a cow, a sheep, a goat, or any other non-human mammal. Preferably, the subject is a human. The term “patient” refers to a human subject.

In embodiments, the subject is a human subject. In one embodiment, the human is an adult human, a pediatric human, or a geriatric human, as those terms are understood by the medical practitioner, for example as defined by the U.S. Food and Drug Administration.

As used herein, the term “pharmaceutically acceptable salt,” refers to a pharmaceutically acceptable base addition salt which are those salts which retain the biological effectiveness and properties of the free acid counterions and which are not biologically or otherwise undesirable compared to the free acid. These salts are prepared from addition of an inorganic base or an organic base to the free acid. Illustrative salts include, but are not limited, to salts derived from inorganic bases include, but are not limited to, the sodium, potassium, lithium, ammonium, calcium, magnesium, iron, zinc, copper, manganese, and aluminum salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, trimethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

The term “polymorph” in the present disclosure refers to a solid crystalline form of a compound described here. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

The term “hydrate” in the present disclosure refers to a compound described here further including a stoichiometric or non-stoichiometric amount of water bound by non-covalent intermolecular forces.

The term “solvate” or “pharmaceutically acceptable solvate,” in the present disclosure refers to a solvate formed from the association of one or more solvent molecules to one of the compounds disclosed herein. The term solvate includes hydrates (e.g., hemi-hydrate, mono-hydrate, dihydrate, trihydrate, tetrahydrate, and the like). In certain embodiments, the solvate is a form of salt bound by a non-covalent bond to another molecule (such as a polar solvent). Such solvates are typically crystalline solids having a substantially fixed molar ratio of solute and solvent. When the solvent is water, the solvate formed is a hydrate. Example hydrates include hemihydrates, mono hydrates, dihydrates, etc.

In embodiments, the invention provides a crystalline form of a compound described herein. In one embodiment, the invention provides a polymorph of an ionic salt described herein.

Certain compounds of the present disclosure possess asymmetric carbon atoms (optical centers) or double bonds; the racemates, diastereomer, geometric isomers, regioisomers and individual isomers (e.g., separate enantiomers) are all intended to be encompassed within the scope of the present disclosure. In embodiments, the compounds of the present disclosure are a particular enantiomer, anomer, or diastereomer substantially free of other forms.

### Methods of Treating Immune-mediated Inflammatory Diseases

The present disclosure provides methods for treating immune-mediated inflammatory diseases (IMIDs) in a subject in need of such treatment. In embodiments, the methods comprising administering to the subject a pharmaceutical composition comprising a resolvin, or a salt thereof, in a dosing regimen of once daily or less than daily, for example one or more times per week, such as once every two days (Q2D), once every three days (Q3D), once every six days (Q6D) or once every seven days (Q7D). In embodiments, the dosing regimen may be at weekly intervals, for example once every two weeks (Q14D), once every three weeks (Q21D) or once every four weeks (Q28D). In accordance with the methods described here, the resolvin, or its salt, is administered either alone as monotherapy, or in combination therapy with one or more additional therapeutic agents. In embodiments, the combination therapy is an adjunctive therapy, for example as an adjunct to biological therapy, non-steroidal anti-inflammatory drugs (NSAIDS), glucocorticoids, disease-modifying antirheumatic drugs (DMARDs), immunosuppressants, aminosalicylates, immunotherapy, hormone therapy, chemotherapy, targeted therapy, gene therapy, or microbiome therapy. The terms “therapy” and “therapies” refer to any method, protocol and/or agent that can be used in the treatment or management of the disease or disorder.

The inflammatory response in humans has two phases: initiation and resolution. At the cellular level, initiation is a protective response characterized by the production and release of pro-inflammatory mediators to activate the innate immune response, resulting in an influx of polymorphonuclear cells (PMNs) from blood to kill and clear foreign invaders and damaged cells. This initiation phase is an active process driven by metabolites of arachidonic acid including the prostaglandins, which are chemo-attractants for eosinophils, neutrophils and monocytes, and the leukotrienes, which elicit adhesion, chemotaxis, and aggregation of leukocytes.

During the peak of inflammation, as the inflammatory stimulus is being reduced, the resolution phase begins, marked by a reduction of pro-inflammatory signals and a switch from the production and release of pro-inflammatory mediators to the production and release of SPMs, including resolvins, protectins, lipoxins, and maresins. SPMs activate and coordinate the resolution phase by halting PMN infiltration and stimulating the recruitment and activation of monocytes and pro-resolving macrophages to clear the tissue of cellular debris in a non-phlogistic manner, enabling the return to tissue homeostasis (Serhan et al., Cold Spring Harb Perspect Biol 2015; 7:a016311).

Unresolved inflammation is widely recognized as a unifying aspect of many chronic diseases and disorders. Accordingly, the ability to resolve excessive inflammation is of paramount importance to human health (Serhan, C. N., Nature 2014 510:92-101; Coussens et al. Nature 2002; 420, 860-867, Grivennikov et al., Cell. 2010; 140(6):883-99, Todoric et al., Cancer Prev Res. 2016; 9(12):895-905; Fishbein et al., Pharmacol Ther. 2021; 218:107670).

In embodiments, provided here are methods for treating immune-mediated inflammatory diseases (IMIDs) in a subject in need of such treatment, including gastrointestinal diseases and disorders, inflammatory disorders, infectious diseases and disorders, metabolic diseases and disorders, neurological disorders, pulmonary diseases and disorders, and cancer, the methods comprising administering to the subject a pharmaceutical composition comprising a resolvin, or a salt thereof, in a dosing regimen of once daily or less than daily, for example one or more times per week, such as once every two days (Q2D), once every three days (Q3D), once every six days (Q6D) or once every seven days (Q7D).

### Gastrointestinal Diseases and Disorders

In embodiments of the methods described here, the gastrointestinal disease or disorder is selected from ulcerative colitis, Crohn's disease, proctitis, pouchitis, eosinophilic colitis, lymphocytic colitis, collagenous colitis, diversion colitis, chemical colitis, ischemic colitis, infectious colitis, pseudomembranous colitis, checkpoint inhibitor-induced colitis, and indeterminate colitis. In embodiments, the gastrointestinal disease or disorder is selected from ulcerative colitis, and Crohn's disease.

In embodiments, the gastrointestinal disease or disorder is selected from bowel obstruction, chronic pancreatitis, colitis, colon cancer, congenital gastrointestinal anomalies, gastroschisis, gastric cancer, esophageal cancer, high-output fistula, parenteral nutrition associated liver disease, postoperative ileus, postoperative intestinal inflammation, short bowel syndrome, and sporadic polyposis. In embodiments, the gastrointestinal disease or disorder is selected from eosinophilic esophagitis, Behcet's disease, irritable bowel syndrome, celiac disease, Intestinal mucositis, NSAID enteropathies, enteric infections, diverticulosis, diverticulitis, gastritis, pancreatitis, viral gastroenteritis, and Whipple's disease.

In embodiments, the gastrointestinal disease or disorder is postoperative intestinal inflammation, postoperative ileus, or a combination thereof. In embodiments, the gastrointestinal inflammatory disease or disorder is postoperative ileus (POI).

In embodiments, the gastrointestinal disease or disorder is inflammatory bowel disease (referred herein as “IBD”), which is the term given for two conditions, Crohn's disease and ulcerative colitis, both characterized by chronic inflammation of the gastrointestinal tract. Crohn's disease may affect any portion of the gastrointestinal tract, including the oral cavity and anus, but most often affects the lower gastrointestinal tract, especially the ileum and/or colon. Ulcerative colitis affects the colon and rectum. The terms “large intestine” and “colon” are used interchangeably in the present disclosure.

Standard of care treatment of mild-to-moderate IBD is aimed at achieving and maintaining remission by controlling inflammation in the target tissues of the gastrointestinal tract. In adults, 5 aminosalicylates (5-ASA), alone or in combination with corticosteroids may be used to induce remission. Generally, corticosteroid treatment is administered for short-term control of symptoms, e.g., to treat acute flare-ups and induce remission of disease symptoms. Longer-term, or maintenance therapy, generally involves the use of non-steroidal agents including 5-ASA, and immune modulators such as azathioprine.

Standard of care treatment of moderate-to-severe IBD is also aimed at achieving and maintaining remission by controlling inflammation in the target tissues of the gastrointestinal tract. In adults, anti-TNF therapy using adalimumab, golimumab, or infliximab alone or in combination with a corticosteroid, methotrexate, thiopurine, or 5-ASA may be used to induce remission. For patients with moderate-to-severe IBD that have failed anti-TNF therapy, an IL-12/23 antagonist such as ustekinumab, an integrin receptor antagonist such as vedolizumab, a Sphingosine-1-phosphate receptor modulators such as ozanimod, or a Janus kinase inhibitor such as tofacitinib may be used. Longer-term, or maintenance therapy, generally involves continuation of anti-TNF therapy, with or without a thiopurine, or continuation of the other therapies described above.

In embodiments, the present disclosure provides for treatment of IBD by administering to the subject a pharmaceutical composition containing a complex as described herein either as monotherapy or in a combination therapy with a second IBD therapeutic agent. In embodiments, the second IBD therapeutic agent is selected from a corticosteroid, 5-ASA, azathioprine, anti-TNF therapy, an integrin receptor antagonist, a Sphingosine-1-phosphate receptor modulator, or a Janus kinase inhibitor. In embodiments, the disclosure provides bis RvE1 magnesium di-lysinate for use in a method of treating IBD in combination with an IBD maintenance therapy, optionally 5-ASA, azathioprine, anti-TNF therapy, integrin receptor antagonist, Sphingosine-1-phosphate receptor modulator, or Janus kinase inhibitor. In embodiments, the disclosure provides bis RvE1 magnesium di-lysinate for use in a method of treating IBD in combination with any one or more of 5-ASA, azathioprine, a corticosteroid, anti-TNF therapy, IL-12/23 antagonist, integrin receptor antagonist, Sphingosine-1-phosphate receptor modulator, or Janus kinase inhibitor. In embodiments, the disclosure provides bis RvE1 magnesium di-lysinate for use in a method of treating IBD in combination with a corticosteroid. In accordance with the foregoing embodiments of combination therapy of bis RvE1 magnesium di-lysinate with a corticosteroid, the corticosteroid may be selected from the group consisting of prednisone, prednisolone, budesonide, hydrocortisone, and beclometasone dipropionate, where the dosage form is an oral dosage form, or from hydrocortisone, prednisolone, and budesonide, where the dosage form is adapted for rectal delivery, for example in the form of an enema or suppository. In accordance with the foregoing embodiments of combination therapy of bis RvE1 magnesium di-lysinate with an anti-TNF therapy, integrin receptor antagonist, Sphingosine-1-phosphate receptor modulator, or Janus kinase inhibitor, the second IBD therapeutic agent may be selected from the group consisting of adalimumab, golimumab, infliximab, vedolizumab, ozanimod, etrasimod, tofacitinib or filgotinib.

### Inflammatory Disorders

In embodiments of the methods described here, the inflammatory disease or disorder is selected from the group consisting of acne, adipose tissue inflammation, allograft rejection, arthritis, bacterial infection, bullous pemphigoid, burn wounds, chelitis, chronic pancreatitis, corneal wound, dermatitis, diabetic wounds, dry eye syndrome, eczema, endometriosis, endotoxin shock, epidermolysis, ankylosing spondylitis, bullosa acquisita, glossitis, heart ischemia, HSV-keratitis, hidradenitis suppurativa, IgA-mediated bullous dermatoses, ischemia reperfusion injury, localized aggressive periodontitis, lupus erythematosus, lyme arthritis, macular edema, oral mucositis, osteoarthritis, periodontitis, peritonitis, pemphigus, postoperative pain, postsurgical cognitive decline, pruritus, psoriasis, psoriatic arthritis, pyoderma gangrenosum, retinopathy, rheumatoid arthritis, scleroderma, Sjogren's syndrome, steroid-induced rosacea, stomatitis, systemic inflammatory response syndrome, temporomandibular joint inflammation, and vascular inflammation.

### Infectious Diseases and Disorders Caused by an Infectious Agent

In embodiments of the methods described here, the disease or disorder is caused by an infectious agent, such as a bacterium, a fungus, or a virus. In embodiments, the disease or disorder is a bacterial infection. In embodiments, the bacterial infection is bacterial pneumonia. In embodiments, the bacterial infection is an E. coli infection. In embodiments, the bacterial infection is a Mycobacterium tuberculosis infection. In embodiments, the disease or disorder is a yeast infection. In embodiments, the yeast infection is a Candida yeast infection. In embodiments, the disease or disorder is sepsis. In embodiments, the sepsis is burn wound sepsis. In embodiments, the disorder is sepsis-induced cardiomyopathy.

### Metabolic Diseases and Disorders

In embodiments of the methods described here, the metabolic disease or disorder is abnormal glucose metabolism manifesting in diabetes, including type 2 diabetes, or pre-diabetes, insulin resistance, abnormal lipid metabolism manifesting as hypertriglyceridemia, i.e., elevated triglycerides, mixed dyslipidemia, hypercholesterolemia, fatty liver, and combined abnormal glucose and lipid metabolism manifesting in obesity; or a dyslipidemic disorder selected from hypertriglyceridemia, hypercholesterolemia and mixed dyslipidemias.

In embodiments, the metabolic disease or disorder is insulin resistance, mixed dyslipidemia, nonalcoholic steatohepatitis (NASH), type 2 diabetes, primary biliary syndrome, and primary schlerosing cholangitis.

In accordance with the methods described here, administration may be alone as monotherapy, or in combination therapy with one or more additional therapeutic agents, including but not limited to an antihyperlipidemic agent or an anti-diabetic agent. Antihyperlipidemic agents that may be used include HMG CoA enzyme inhibitors (e.g., statins), cholesterol absorption inhibitors, and cholesterol esterase transfer protein (CETP) inhibitors. In embodiments, the antihyperlipidemic agent is selected from a statin, a cholesterol absorption inhibitor, a CETP inhibitor, and pharmaceutically-acceptable salts and prodrugs of any of the foregoing.

### Neurological Disorders

In embodiments of the methods described here, the neurological diseases and disorders that may be treated include, without limitation, Alzheimer's disease, peripheral nerve injury, amyotrophic lateral sclerosis, multiple sclerosis, pain, and fibromyalgia. In embodiments, the neurological disease or disorder is selected from postoperative delirium, acute postsurgical pain, fibromyalgia, endometriosis, lower genital tract pain, vulvodynia, post-herpetic neuralgia, chronic lower back pain, treatment or management of pain associated with osteoarthritis, diabetic peripheral neuropathy and musculoskeletal injury or trauma. In the context of the methods described here, the term “treating” may refer to the amelioration or stabilization of one or more clinical symptoms associated with the disease or disorder being treated.

In embodiments, the amount is effective to treat one or more symptoms of the neurological disorder.

In embodiments, the neurological disorder is a psychiatric disorder. In embodiments, the psychiatric disorder is selected from attention deficit hyperactivity disorder (ADHD) and depression. In embodiments, the neurological disease or disorder is postoperative cognitive dysfunction (POCD) or postoperative delirium.

The disclosure also provides methods for treating or managing pain. In embodiments, the pain is nociceptive pain and the method comprises administering to a subject in need of treatment for nociceptive pain a pharmaceutical composition comprising an effective amount a compound described here, or mixtures thereof.

In embodiments, the disclosure provides methods for treating or managing pain associated with inflammation, fibromyalgia, endometriosis, vulvodynia, osteoarthritis, diabetic peripheral neuropathy, and musculoskeletal injury or trauma.

In embodiments, the disclosure also provides methods for treating or managing acute postsurgical pain and chronic lower back pain.

### Pulmonary Diseases and Disorders

In embodiments of the methods described here, the pulmonary and vascular diseases and disorders that may be treated include, without limitation, asthma, pulmonary inflammation, bronchiolitis obliterans, bronchopulmonary dysplasia, also referred to as chronic lung disease of infancy, cystic fibrosis, allergic airway response, acute lung injury, acute respiratory distress syndrome, lung injury, idiopathic pulmonary fibrosis, bacterial pneumonia, cigarette smoke-induced lung inflammation, and vascular inflammation.

In the context of the methods described here, the term “treating” may refer to the amelioration or stabilization of one or more clinical symptoms associated with the disease or disorder being treated. In embodiments, treating leads to the elimination of a clinical symptom of the disease or disorder being treated, however, elimination is not required. In embodiments, the severity of the symptom is decreased. In the context of the present disclosure, the term “treating” is not meant to encompass the amelioration or stabilization of clinical symptoms that are not associated with the disease or disorder being treated, and instead, for example, are associated with a treatment modality, such as a side effect or adverse event related to another therapy, unless explicitly stated. In addition, the term “treating” is not intended to encompass prevention, or the administration of a resolvin, or its salt, prior to diagnosis in the subject being treated.

In accordance with the methods described here, a therapeutically effective amount of the resolvin, or a salt thereof, is administered to the subject in need of therapy. The therapeutically effective amount is an amount or dose of the resolvin, or its salt, sufficient to treat the disease or disorder, or sufficient to achieve a desired therapeutic outcome, for example, the amelioration or stabilization of one or more biomarkers of disease progression or one or more clinical symptoms.

In embodiments, the therapeutically effective amount comprises from about 0.1 mg to 100 mg resolvin per dose, preferably between about 0.5 mg and 20 mg, or between about 1 mg and 10 mg resolvin per dose. When administered using continuous dosing, the therapeutically effective amount comprises from about 0.1 mg to 100 mg resolvin per day or per week or per month, preferably between about 0.5 mg and 20 mg per day or per week or per month, or between about 1 mg and 10 mg resolvin per day or per week or per month.

A dose of the resolvin, or its salt, is administered in accordance with a therapeutic regimen as described herein, for example once daily or less than daily (LTD). In embodiments, the regimen may comprise dosing one or more times per week, such as every two days (Q2D), every three days (Q3D), every six days (Q6D) or every seven days (Q7D). In embodiments, the regimen may comprise dosing once or twice a month, such as every two to four weeks. In embodiments, the regimen comprises dosing every 1, 2, 4, 6, or 8 weeks. It is understood that a regimen may include successive periods of different dosing frequency, for example QD for a period of time followed by an LTD dosing regimen, such as Q2D, Q3D, Q6D, Q7D or less frequently than Q7D, or alternatively. Q2D, Q3D, Q6D or Q7D for a period of time followed by dosing less frequently, such as every 2, 4, 6, or 8 weeks, for a second period of time, or alternatively, any LTD dosing regimen for a period of time followed by a dosing regimen of lesser or greater frequency.

In embodiments, the pharmaceutical composition is administered as part of a maintenance therapy every two days (Q2D), every three days (Q3D), every six days (Q6D) or every seven days (Q7D) using intravenous, subcutaneous, intraperitoneal, or other routes of administration during clinic visits; or taken outside of the clinic using routes of administration that can be self-administered by the patient (e.g., subcutaneous, oral, sublingual, inhalable, and topical).

In embodiments, the pharmaceutical composition comprising a resolvin, or a salt thereof, may be administered by any suitable route of administration, including subcutaneous, intravenous, intramuscular, oral, transdermal, sublingual, topical, or by inhalation. In embodiments, administration is by a subcutaneous or intramuscular route. In embodiments, the oral dosage form may be in the form of a tablet, capsule, powder, solution, suspension, or emulsion. In embodiments of subcutaneous dosing, the dosage form may be a liquid, including an aqueous liquid, and the volume of a dose may be between 0.2 mL and 5 mL, preferably between 0.5 mL and 2 mL and preferably no greater than 1 mL. In embodiments of intravenous dosing, a dose may be delivered in the form of a bolus or by infusion, for example pump infusion or drip infusion.

### Combination Therapies

In the context of the methods described above, the methods may further comprise administering a pharmaceutical composition comprising a resolvin, or its salt, in combination therapy, for example in combination with one or more additional therapeutic agents, which may be referred to as active pharmaceutical ingredients (API), or one or more additional therapies, such as surgery, radiation therapy, biological therapy, non-steroidal anti-inflammatory drugs (NSAIDS), glucocorticoids, disease-modifying antirheumatic drugs (DMARDs), immunosuppressants, aminosalicylates, immunotherapy, hormone therapy, CAR T cell therapy, gene therapy, or microbiome therapy. In embodiments, administration of the resolvin, or its salt, may be simultaneous, separate, or sequential with respect to the one or more additional APIs or therapies.

With respect to the foregoing therapeutic regimens for those drugs administered parenterally in a hospital or outpatient setting, typically on a weekly basis one to four times every 28 days, the pharmaceutical composition may be administered on a weekly basis on the same day that the other drugs are administered or off-cycle when other therapies are not given, or QD, Q2D, Q3D, Q6D, Q7D or less frequently at home in between cycles of the other therapy or as maintenance once the other therapies are terminated.

With respect to the foregoing therapeutic regimens, the pharmaceutical composition comprising a resolvin, or its salt, is administered QD, Q2D, Q3D, Q6D or Q7D at home or as co-therapy in the clinic if the resolvin regimen overlaps with the regimen of the primary therapy administered in the clinic to optimize patient adherence.

As used herein, “combination therapy” or “co-therapy” includes the administration of a therapeutically effective amount of one or more of the compounds or compositions described here as part of a specific treatment regimen intended to provide a beneficial effect from the co-action of the compounds described here and one or more additional active agents (i.e., additional API) or therapies. The beneficial effect of the combination in the context of the present methods may include, for example, pharmacokinetic or pharmacodynamic co-action resulting from the combination. The beneficial effect of the combination may also relate to the mitigation of a toxicity, side effect, or adverse event associated with another agent in the combination. “Combination therapy” is not intended to encompass the administration of two or more compounds as part of separate monotherapy regimens that incidentally and arbitrarily result in a beneficial effect that was not intended or predicted. In some embodiments, the compounds and compositions described here are useful as adjunctive therapy to a primary therapy.

The one or more additional APIs can be formulated for co-administration with a resolvin or its salt form as described herein, either in a single dosage form, or in separate dosage forms. When the additional API is administered separately from the resolvin, or its salt, it can be administered by the same or a different route of administration from the resolvin. Accordingly, the methods described here encompass administering the resolvin, or its salt, together with the at least one additional API, or separately from the additional API. Where delivery is together, the resolvin, or its salt, may be delivered in as a single dosage form that includes the additional API, or in a separate dosage form.

In some aspects, the administration of a composition comprising an resolvin or its salt in combination with one or more additional therapeutic agents as described herein provides a synergistic response in the subject being treated. In this context, the term “synergistic” refers to the efficacy of the combination being more effective than the additive effects of either single therapy alone or less variable than either single therapy alone. The synergistic effect of a combination therapy according to the disclosure can permit the use of lower dosages and/or less frequent administration of at least one agent in the combination compared to its dose and/or frequency outside of the combination. Additional beneficial effects of the combination can be manifested in the longer therapeutic use of at least one agent in the combination compared to its duration of use without the combination or avoidance or reduction of adverse or unwanted side effects associated with the use of either therapy in the combination alone (also referred to as monotherapy).

### Salt Compounds

In certain embodiments, the compounds for use in the compositions and methods described here are salts of resolvins in which at least one or two resolvin molecules are ionically bound to at least one basic function that is provided by a scaffold as described in Formulas I-IV below. In general, the carboxylic acid moiety of the resolvin molecule or molecules forming the SPM component of the compounds described here is deprotonated to form an ionic bond with a basic function (or functions) of the scaffold portion of the compound.

The compounds described herein can exist in unsolvated forms as well as solvated forms, including hydrated forms. In general, the solvated forms are equivalent to unsolvated forms and are intended to be encompassed within the scope of the present invention. In general, all physical forms are equivalent for the uses contemplated by the present invention and are intended to be within the scope of the present invention.

The abbreviations used herein have their conventional meaning within the chemical and biological arts. The chemical structures and formulae set forth herein are constructed according to the standard rules of chemical valency known in the chemical arts. For example, in instances where a substituents such as —NH₃ are shown without a charge, it is understood to possess a formal charge, i.e. NH₃⁺.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons).

The term “basic function” refers to a positively charged or protonated primary amine, a positively charged secondary amine, a positively charged tertiary amine, or a positively charged guanidine. In embodiments, basic function refers to —NH₃⁺, —NHC(NH₂⁺)NH₂, —NHR⁶R⁷, —NR⁶R⁷R⁸, wherein R⁶, R⁷, and R⁸ are each independently hydrogen, —CN, —COOH, —CONH₂, unsubstituted alkyl, unsubstituted heteroalkyl, unsubstituted cycloalkyl, unsubstituted heterocycloalkyl, unsubstituted aryl, or unsubstituted heteroaryl; R⁶ and R⁷ substituents bonded to the same nitrogen atom may optionally be joined to form a unsubstituted heterocycloalkyl or unsubstituted heteroaryl. In embodiments, the basic function is a hydrogen bond acceptor. In embodiments, the basic function is a positively charged amine.

It is understood that due to resonance a charge may be distributed across the molecule. The chemical structures and formulae set forth herein are constructed according to the standard rules of chemical valency known in the chemical arts, and as such one of skill in the art would recognize the equivalency of the moieties possessing resonance structures.

In embodiments, the “side chain of an amino acid” or “side chain” or “side-chain” as used herein is used in accordance with its ordinary meaning and refers to the functional substituent contained on naturally occurring amino acids. Naturally occurring amino acids are those encoded by the genetic code (e.g. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the side chain of an amino acid is ionized (e.g., it has a formal charge).

In embodiments, the side chain is selected from the group consisting of H,

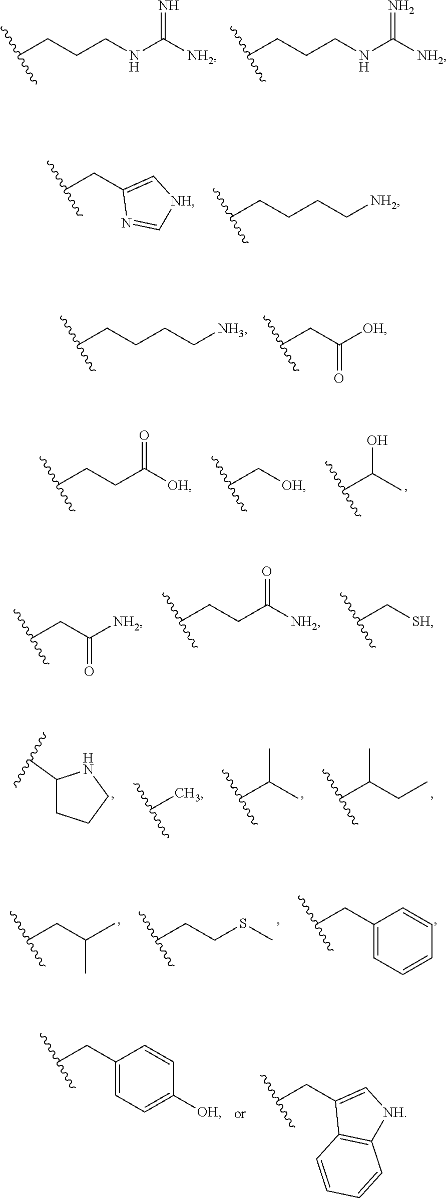

In embodiments, the side chain is H. In embodiments, the side chain is

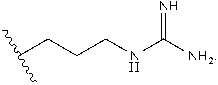 [SMILES: *CCCNC(=N)N]

In embodiments, the side chain is

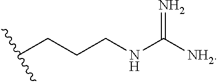 [SMILES: *CCCNC(=N)N]

In embodiments, the side chain is

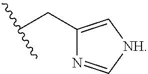 [SMILES: *Cc1c[nH]cn1]

In embodiments, the side chain is

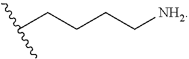 [SMILES: *CCCCN]

In embodiments, the side chain is

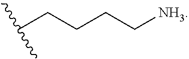

In embodiments, the side chain is

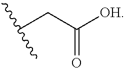 [SMILES: *CC(=O)O]

In embodiments, the side chain is

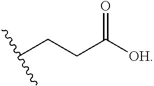 [SMILES: *CCC(=O)O]

In embodiments, the side chain is

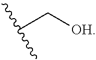 [SMILES: *CO]

In embodiments, the side chain is

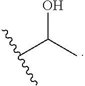 [SMILES: *C(C)O]

In embodiments, the side chain is

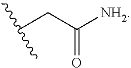 [SMILES: *CC(N)=O]

In embodiments, the side chain is

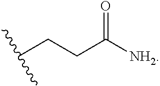 [SMILES: *CCC(N)=O]

In embodiments, the side chain is

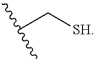 [SMILES: *CS]

In embodiments, the side chain is

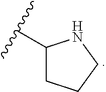 [SMILES: *C1CCCN1]

In embodiments, the side chain may optionally be joined to an adjacent nitrogen to form a unsubstituted heterocycloalkyl (e.g., pyyrolidinyl).

In embodiments, the side chain is

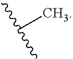

In embodiments, the side chain is

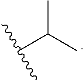 [SMILES: *C(C)C]

In embodiments, the side chain is

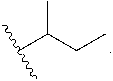 [SMILES: *C(C)CC]

In embodiments, the side chain is

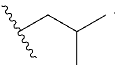 [SMILES: *CC(C)C]

In embodiments, the side chain is

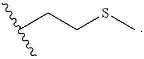 [SMILES: *CCSC]

In embodiments the side chain is

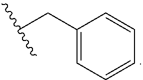 [SMILES: *Cc1ccccc1]

In embodiments, the side chain is

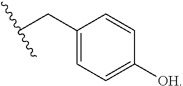 [SMILES: *Cc1ccc(O)cc1]

In embodiments, the side chain is

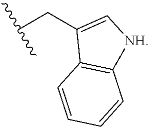 [SMILES: *Cc1c[nH]c2ccccc12]

The side chain of glycine is H. The side chain of arginine is

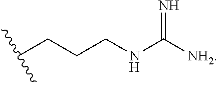 [SMILES: *CCCNC(=N)N]

The side chain of histidine is

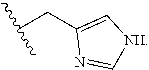 [SMILES: *Cc1c[nH]cn1]

The side chain of lysine is

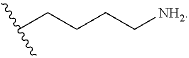 [SMILES: *CCCCN]

The side chain of aspartic acid is

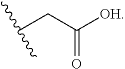 [SMILES: *CC(=O)O]

The side chain of glutamic acid is

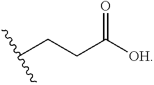 [SMILES: *CCC(=O)O]

The side chain of serine is

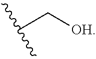 [SMILES: *CO]

The side chain of threonine is

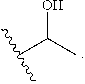 [SMILES: *C(C)O]

The side chain of asparagine is

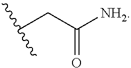 [SMILES: *CC(N)=O]

The side chain of glutamine is

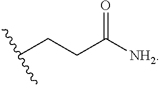 [SMILES: *CCC(N)=O]

The side chain of cysteine is

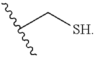 [SMILES: *CS]

The side chain of proline is

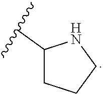 [SMILES: *C1CCCN1]

The side chain of alanine is

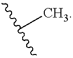

The side chain of valine is

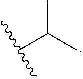 [SMILES: *C(C)C]

The side chain of isoleucine is

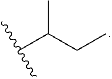 [SMILES: *C(C)CC]

The side chain of leucine is

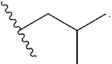 [SMILES: *CC(C)C]

The side chain of methionine is

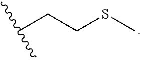 [SMILES: *CCSC]

The side chain of phenylalanine is

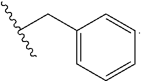 [SMILES: *Cc1ccccc1]

The side chain of tyrosine is

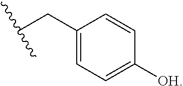 [SMILES: *Cc1ccc(O)cc1]

The side chain of tryptophan is

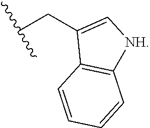 [SMILES: *Cc1c[nH]c2ccccc12]

The term “non-natural amino acid side-chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride, cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-3-(2-quinolyl)-Ala-OH, N-Boc-1,2,3,6-tetrahydro-2-pyridinecarboxylic acid, Boc-3-(4-thiazolyl)-Ala-OH, Boc-3-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-3-(4-thiazolyl)-Ala-OH, Fmoc-3-(2-thienyl)-Ala-OH, 4-(Hydroxymethyl)-D-phenylalanine.

### Formula I Compounds

In embodiments, the disclosure provides compounds of Formula I, including enantiomers, polymorphs, solvates, and hydrates thereof:

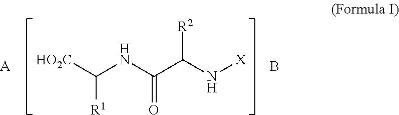

wherein
A and B are each independently resolvin anion;
A and B may be the same or different;
either A or B, but not both, may be absent,
R¹ and R² are each independently a C₁-C₁₀ alkyl comprising at least one basic function and is optionally branched;
X is H or CO—Z and Z is a single amino acid residue or a peptide comprising 2 to 18 amino acid residues;
when either A or B is absent:
one of R¹, R² and CO—Z is protonated; or
H is positively charged; and
the one of R¹, R² and the CO—Z that is protonated or the positively charged H forms an ionic bond with either A or B; and
when A and B are both present:
two of R¹, R² and CO—Z are protonated; or
one of R¹, R² and CO—Z is protonated, and H is positively charged; and
the two of R¹, R² and the CO—Z that are protonated or the one of R¹, R² and the CO—Z that is protonated and the positively charged H each respectively form an ionic bond with A and B.

Compounds of Formula I comprise a peptide component consisting of at least 2 amino acid moieties and one or two resolvin molecules (A, B) as the SPM component. The SPM component is described in more detail below. In embodiments, the SPM component comprises or consists of an SPM selected from an E or D series resolvin. In embodiments, the SPM component comprises or consists of an aspirin-triggered (AT) resolvin. In embodiments, the AT resolvin is selected from the group consisting of AT-RvE1, AT-RvE2, and AT-RvE3. In embodiments, the AT resolvin is selected from the group consisting of AT-RvE1, AT-RvD1, and AT-RvD2. In embodiments, the SPM component consists of an E series resolvin; in embodiments, the E series resolvin is selected from RvE1, RvE2, RvE3, RvE4, AT-RvE1, AT-RvE2, and AT-RvE3.

The peptide component may be from 2 to 10 or 2 to 20 amino acids in length, preferably 2, 3, 4, or 5 amino acids in length. The peptide component consists of 2 amino acid residues when X is H, or is a peptide of from 3 to 5, 3 to 10, or 3 to 20 amino acid residues where X is CO—Z.

Each amino acid moiety of the peptide component may, independently, comprise or consist of a single natural or non-naturally occurring amino acid residue. In embodiments, the amino acid residues are independently selected from a residue of glycine, alanine, valine, leucine, isoleucine, serine, cysteine, threonine, methionine, proline, phenylalanine, tyrosine, tryptophan, histidine, lysine, arginine, aspartic acid, glutamic acid, asparagine, and glutamine.

R¹ and R² are each independently unsubstituted C₁-C₁₀ alkyl including at least one basic function. In embodiments, the basic function is the side chain of an amino acid moiety. In embodiments, the amino acid moiety is selected from lysine, arginine, and glutamine. In embodiments, the basic function is selected from the group consisting of a positively charged primary amine, a positively charged secondary amine, a positively charged tertiary amine, and a positively charged guanidine.

In embodiments, basic function refers to —NH₃⁺, —NHC(NH₂⁺)NH₂, —NHR⁶R⁷, or —NR⁶R⁷R⁸, wherein R⁶, R⁷, R⁸ are each independently hydrogen, —CN, —COOH, —CONH₂, unsubstituted alkyl, unsubstituted heteroalkyl, unsubstituted cycloalkyl, unsubstituted heterocycloalkyl, unsubstituted aryl, or unsubstituted heteroaryl; R⁶ and R⁷ substituents bonded to the same nitrogen atom may optionally be joined to form a unsubstituted heterocycloalkyl or unsubstituted heteroaryl. In embodiments, the basic function is a positively charged amine. In embodiments, the basic function is a primary amine. In embodiments, the basic function is —NH₃⁺.

In embodiments, X is H and the peptide component consists of a dipeptide of amino acids independently selected from lysine, arginine, and glutamine, or a derivative of one or more of the foregoing. In embodiments, X is H and the peptide component consists of a dipeptide of lysine. In embodiments, X is H and R¹ and R² are each independently selected from —(CH₂)₃—NHC(NH₂⁺)—NH₂, —(CH₂)₄—NH₃⁺, and —(CH₂)₂—C(O)NH₃⁺. In embodiments, R¹ and R² are the same. In embodiments, R¹ and R² are different.

In embodiments, X is CO—Z, and Z is either a single amino acid residue or a peptide of from 2 to 10 or 2 to 5 amino acid residues, and the peptide component comprises at least one or two amino acids independently selected from lysine, arginine, and glutamine.

In embodiments, X is H and R¹ and R² are each independently selected from —(CH₂)₃—NHC(NH₂⁺)NH₂, —(CH₂)₄—NH₃⁺, and —(CH₂)₂—C(O)NH₃. In embodiments, R¹ and R² are the same. In embodiments, R¹ and R² are different.

In embodiments, X is CO—Z, and Z is either a single amino acid residue or a peptide of from 2 to 10 or 2 to 5 amino acid residues, and R¹ and R² are each independently selected from —(CH₂)₃—NHC(NH₂⁺)NH₂, —(CH₂)₄—NH₃⁺, and —(CH₂)₂—C(O)NH₃⁺. In embodiments, R¹ and R² are the same. In embodiments, R¹ and R² are different.

In embodiments, —NHC(NH₂⁺)NH₂ is 2

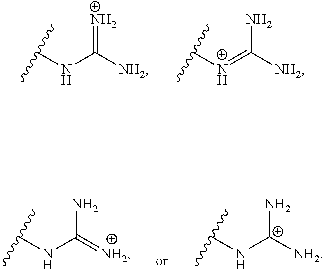

In embodiments, either A or B is absent. Where either A or B is absent, the compound may be referred to as “mono” salt. In embodiments, A and B are both present. Where A and B are both present, the compound may be referred to as a “bis” salt. In one embodiment, A and B are each an SPM, and A and B are the same or different.

In embodiments, A and B are the same or different and each is independently selected from an E series resolvin. In embodiments, the E series resolvin is selected from RvE1, RvE2, RvE3, RvE4, AT-RvE1, AT-RvE2, and AT-RvE3. In embodiments, A and B are the same and selected from the group consisting of resolvin E1, resolvin D2, and aspirin triggered resolvin D1.

In embodiments, A and B are the same and selected from the group consisting of RvE1, RvE2, RvE3, RvE4, AT-RvE1, AT-RvE2, and AT-RvE3; R¹ and R² are both —(CH₂)₄—Y², Y² is NH₃⁺, and X is H. This selection of R¹, R², and Y² may be referred to herein as a “lysyl lysine” (which may be abbreviated herein as “lys-lys”) dipeptide. In this embodiment, the peptide component is a lysine dipeptide.

In embodiments, A and B are the same and selected from an E or D series resolvin; R¹ and R² are both —(CH₂)₄—Y², Y² is NH₃⁺, and X is H. As noted above, this selection of R¹, R², and Y² may be referred to herein as a “lysyl lysine” dipeptide or a “lys-lys” dipeptide. In this embodiment, the peptide component is a lysine dipeptide.

In embodiments, the compound of Formula I is a mono or bis SPM lysyl-lysine (lys-lys) compound selected from the group consisting of RvE1 lys-lys, RvE2 lys-lys, RvE3 lys-lys, RvE4 lys-lys, AT-RvE1 lys-lys, AT-RvE2 lys-lys, and AT-RvE3 lys-lys. In embodiments, the compound of Formula I is a mono or bis SPM lysyl-lysine (lys-lys) compound selected from the group consisting of RvD1 lys-lys, RvD2 lys-lys, RvD3 lys-lys, RvD4 lys-lys, RvD5 lys-lys, RvD6 lys-lys, and the aspirin-triggered counterparts of the foregoing.

Exemplary compounds of the lysyl-lysine embodiment of Formula I are provided in Table 4. In embodiments, the compound of Formula I is selected from the group consisting of Compounds 4, 9, 44, 49, 54, and 59 (E series) of Table 4. In embodiments, a compound of Formula I is selected from the group consisting of Compounds 4, 9, 14, 19, 24, 29, 34, and 39 of Table 4. In embodiments, a compound of Formula I is selected from the group consisting of Compounds 4, 9, 24, 29, 34, and 39 of Table 4. In embodiments, a compound of Formula I is selected from the group consisting of Compounds 4 and 9 (RvE1 and AT-RvE1 embodiments).

### Formula II Compounds

In embodiments, the disclosure provides compounds of Formula II or an enantiomer, polymorph, solvate, or hydrate thereof:

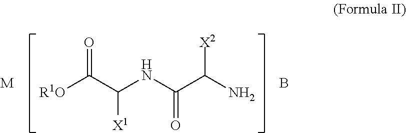

wherein R¹ is H, or absent, X¹ and X² are each independently the side chain of an amino acid residue, M is a positively charged optional molecule, and B is a resolvin anion.

In embodiments, R¹ is H and X¹ and X² are the side chain of glycine.

In embodiments, R¹ is H and X¹ is the side chain of lysine, and X² is selected from the side chain of valine, the side chain of serine, the side chain of leucine, the side chain of histidine

A compound of Formula II consists of at least (i) a dipeptide component and (ii) an SPM component (B), with a positively charged optional molecule (M). The dipeptide component contains X¹ and X² which may be the same or different, and are each the side chain of an amino acid residue. In embodiments, at least one of X¹ and X² is the side chain of an amino acid residue selected from serine, threonine, glycine, alanine, valine, leucine, isoleucine, methionine, and phenylalanine. In embodiments, where one of X¹ and X² is the side chain of an amino acid residue selected from serine, threonine, glycine, alanine, valine, leucine, isoleucine, methionine, and phenylalanine, the remainder of X¹ or X² is the side chain of an amino acid independently selected from lysine, arginine, histidine, aspartate, glutamate, serine, threonine, asparagine, glutamine, cysteine, glycine, proline, alanine, valine, isoleucine, leucine, methionine, phenylalanine, tyrosine, and tryptophan. In embodiments, the remainder is the side chain of lysine. In embodiments, at least one of X¹ and X² is the side chain of glycine, valine, serine, leucine, or histidine, and the remainder is the side chain of lysine.

In embodiments, the SPM component (B) comprises or consists of an E or D series resolvin. In embodiments of the compound of Formula II, B is selected from the group consisting of RvE1, RvE2, RvE3, RvE4, AT-RvE1, AT-RvE2, and AT-RvE3. In embodiments, the SPM component (B) comprises or consists of a D series resolvin selected from the group consisting of RvD1, RvD2, RvD3, RvD4, RvD5, RvD6, and their aspirin-triggered counterparts. In embodiments, the SPM component comprises or consists of an aspirin-triggered (AT) resolvin.

In embodiments, the compound of Formula II is a glycine dipeptide where R¹ is H, X¹ and X² are each H, M is absent and B is selected from the group consisting of RvE1, RvE2, RvE3, RvE4, AT-RvE1, AT-RvE2, and AT-RvE3.

In embodiments, the compound of Formula II is a glycine dipeptide where R¹ is H, X¹ and X² are each H, M is absent and B is selected from the group consisting of RvD1, RvD2, RvD3, RvD4, RvD5, RvD6, and their aspirin-triggered counterparts.

The positively charged optional molecule (M) has at least one basic function which forms an ionic bond with the terminal carboxyl of the amino acid component. In embodiments, M is a monovalent metal cation, e.g., Na⁺, K⁺, or a molecule having at least one basic function, such as a monovalent amine-based cation, e.g., tri-ethanolamine, or tri-ethylamine, or a basic pharmaceutical compound such as metformin or gabapentin.

Compounds of Formula II encompass simple salts of dipeptides and an SPM component (Formula IIa), simple metal salts of the dipeptides and an SPM component with a monovalent metal (Formula IIb), and simple non-metal salts of the dipeptides and an SPM component with a non-metal molecule having at least one basic function (Formula IIc).

### Formula III Compounds

In embodiments, the disclosure provides compounds of Formula III or an enantiomer, polymorph, solvate, or hydrate thereof:

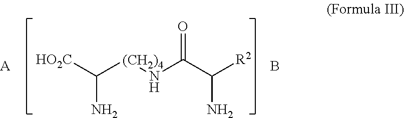

wherein
R² is a C₁-C₁₀ alkyl comprising at least one basic function;
A and B are each independently a resolvin anion;
A and B may be the same or different; and
either A or B, but not both, may be absent.

In embodiments, R² is the side chain of an amino acid residue selected from lysine, arginine, and glutamine. In embodiments, R² is the side chain of lysine. In embodiments, R² is selected from the group consisting of —(CH₂)₃—NHC(NH₂⁺)NH₂, —(CH₂)₄—NH₃⁺, and —(CH₂)₂—C(O)NH₃⁺. In embodiments, R² is —(CH₂)₄—NH₃⁺.

In embodiments, the basic function of R² is selected from the group consisting of a positively charged primary amine, a positively charged secondary amine, a positively charged tertiary amine, and a positively charged guanidine.

In embodiments, the basic function of R² refers to —NH₃⁺, —NHC(NH₂⁺)NH₂, —NHR⁶R⁷, or —NR⁶R⁷R⁸, wherein R⁶, R⁷, R⁸ are each independently hydrogen, —CN, —COOH, —CONH₂, unsubstituted alkyl, unsubstituted heteroalkyl, unsubstituted cycloalkyl, unsubstituted heterocycloalkyl, unsubstituted aryl, or unsubstituted heteroaryl; R⁶ and R⁷ substituents bonded to the same nitrogen atom may optionally be joined to form a unsubstituted heterocycloalkyl or unsubstituted heteroaryl. In embodiments, the basic function is a positively charged amine. In embodiments, the basic function is a primary amine. In embodiments, the basic function is —NH₃⁺.

In embodiments, R² is the side chain of lysine, A and B are the same molecule and are selected from an E series resolvin; in embodiments, the E series resolvin is selected from the group consisting of RvE1, RvE2, RvE3, RvE4, AT-RvE1, AT-RvE2, and AT-RvE3. In embodiments, R² is the side chain of lysine, A and B are the same molecule and are selected from the group consisting of RvD1, RvD2, RvD3, RvD4, RvD5, RvD6, and their aspirin-triggered counterparts.

In embodiments, the SPM component (A, B) comprises or consists of an E series resolvin; in embodiments, the E series resolvin is selected from the group consisting of RvE1, RvE2, RvE3, RvE4, AT-RvE1, AT-RvE2, and AT-RvE3. In embodiments, the SPM component (A, B) comprises or consists of a D series resolvin selected from RvD1, RvD2, RvD3, RvD4, RvD5, RvD6, and their aspirin-triggered counterparts.

In an embodiment of the compound of Formula III, A and B are the same or different and each is independently selected from an E series resolvin and R² is —(CH₂)₄—NH₃⁺. In embodiments, the E series resolvin is selected from RvE1, RvE2, RvE3, RvE4, AT-RvE1, AT-RvE2, and AT-RvE3. This selection of R² may be referred to herein as a “linear lysyl lysine” (linear lys-lys) dipeptide. In this embodiment, the peptide component is a lysine dipeptide. In embodiments, A and B are the same.

In an embodiment of the compound of Formula III, A and B are the same and selected from a D series resolvin selected from RvD1, RvD2, RvD3, RvD4, RvD5, RvD6, and their aspirin-triggered counterparts, and R² is —(CH₂)₄—NH₃⁺. This selection of R² may be referred to herein as a “linear lysyl lysine” (linear “lys-lys”) dipeptide. In this embodiment, the peptide component is a lysine dipeptide.

Exemplary compounds of Formula III are provided in Table 4. In embodiments, the compound of Formula III is selected from the group consisting of Compounds 5, 10, 45, 50, 55, and 60 (E series) of Table 4. In embodiments, a compound of Formula III is selected from the group consisting of Compounds 5, 10, 15, 20, 25, 30, 35, and 40 of Table 4. In embodiments, a compound of Formula III is selected from the group consisting of Compounds 5 and 10 (RvE1 and AT-RvE1 embodiments) of Table 4.

### Formula IV Compounds

In embodiments, the disclosure provides compounds of Formula IV or an enantiomer, polymorph, solvate, or hydrate thereof:

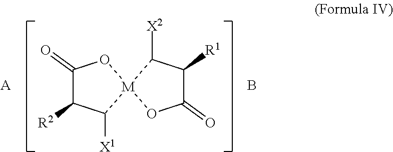

wherein
M is a divalent metal;
A and B are each independently a resolvin anion;
A and B may be the same or different;
either A or B, but not both, may be absent;
R¹ and R² are each independently a C₁-C₁₀ alkyl comprising at least one basic function;
X¹ and X² are each independently H or CO—Z and Z is a peptide comprising 1 to 5 amino acids or a pharmaceutically acceptable salt thereof;
when either A or B is absent:
one of R¹, R² and the two CO—Z's is protonated; or
one of the two H's is positively charged; and
the one of R¹, R² and the two CO—Z's that is protonated or the one of the positively charged H's forms an ionic bond with either A or B; and
when A and B are both present:
two of R¹, R² and the two CO—Z's are protonated; or
one of R¹, R² and the two CO—Z's is protonated, and one of the two H's is positively charged; and
the two of R¹, R² and the two CO—Z's that are protonated or the one of R¹, R² and the two CO—Z's that is protonated and the positively charged H each respectively form an ionic bond with A and B.

Compounds of Formula IV have two amino acid moieties coordinated around a divalent metal cation as the amino acid component and one or two resolvin molecules as the SPM component. In embodiments, the divalent metal cation is Mg²⁺, Ca²⁺, Mn²⁺, Fe²⁺, Cu²⁺, Co²⁺, Ni²⁺, Mo²⁺ or Zn²⁺. In embodiments, the divalent metal cation is Mg²⁺. In embodiments, the divalent metal cation is Ca²⁺. In embodiments, the divalent metal cation is Zn²⁺.

In embodiments, the amino acid component includes or consists of lysine or arginine. In embodiments, the amino acid component includes lysine or arginine. In embodiments, the basic function of R¹ and R² is selected from a primary amine, a secondary amine, a tertiary amine, and a guanidine. In embodiments, basic function refers to —NH₃⁺, —NHC(NH₂⁺)NH₂, —NHR⁶R⁷, or —NR⁶R⁷R⁸, wherein R⁶, R⁷, R⁸ are each independently hydrogen, —CN, —COOH, —CONH₂, unsubstituted alkyl, unsubstituted heteroalkyl, unsubstituted cycloalkyl, unsubstituted heterocycloalkyl, unsubstituted aryl, or unsubstituted heteroaryl; R⁶ and R⁷ substituents bonded to the same nitrogen atom may optionally be joined to form a unsubstituted heterocycloalkyl or unsubstituted heteroaryl. In embodiments, the basic function is a hydrogen bond acceptor. In embodiments, the basic function is a hydrogen bond donor. In embodiments, the basic function is a positively charged amine.

In embodiments, R¹ and R² are each the side chain of an amino acid residue having a basic function. In embodiments, R¹ and R² are the same and the amino acid residue is lysine or arginine.

In embodiments, R¹ and R² are independently selected from —(CH₂)₃—Y¹, and —(CH₂)₄—Y², where Y¹ and Y² are each a basic function which may be the same or different. In embodiments, R¹ is —CH₂CH₂NH₃. In embodiments, R² is —CH₂CH₂NH₃. In embodiments, R¹ is —CH₂CH₂CH₂CH₂NH₃. In embodiments, R² is —CH₂CH₂CH₂CH₂NH₃.

In embodiments, R¹ and R² are both —(CH₂)₄—Y², and Y² is —NH₃⁺.

In embodiments, R¹ and R² are both —(CH₂)₃—Y¹, and Y¹ is —NHC(NH₂⁺)NH₂.

In embodiments, R¹ is —(CH₂)₃—Y¹, Y¹ is —NHC(NH₂⁺)NH₂, Y² is —(CH₂)₄—Y², and Y² is —NH₃⁺. In embodiments, R¹ is —(CH₂)₄—Y², Y² is —NH₃⁺, R² is —(CH₂)₃—Y¹, and Y¹ is NHC(NH₂⁺)NH₂.

In embodiments, X¹ and X² are the same and are hydrogen (H). In embodiments, X¹ is hydrogen. In embodiments, X² is hydrogen.

In an embodiment of the compound of Formula IV, A and B are the same or different and each is independently selected from an E series resolvin. In embodiments, the E series resolvin is selected from RvE1, RvE2, RvE3, RvE4, AT-RvE1, AT-RvE2, and AT-RvE3, M is Mg²⁺, Ca²⁺, or Zn²⁺, R¹ and R² are both —(CH₂)₄—Y² and Y² is NH₃⁺; and X¹ and X² are H. This selection of R¹, R², and Y² may be referred to herein as the metal “di-lysinate”, e.g., “magnesium di-lysinate” or “Mg-di-lysinate”. In Table 4, the metal di-lysinate name is abbreviated “SPM-Mlys” where “M” is the metal, e.g., Mg, Ca, or Zn. In this embodiment, the peptide component consists of a lysine dipeptide. In embodiments, A and B are the same.

In an embodiment of the compound of Formula IV, A and B are the same and selected and E or D series resolvin; M is Mg²⁺, Ca²⁺, or Zn²⁺, R¹ and R² are both —(CH₂)₄—Y² and Y² is NH₃⁺; and X¹ and X² are H. This selection of R¹, R², and Y² may be referred to herein as the metal “di-lysinate”, e.g., “magnesium di-lysinate” or “Mg-di-lysinate”. In this embodiment, the peptide component consists of a lysine dipeptide.

In embodiments, the SPM component (A,B) comprises or consists of an E series resolvin. In embodiments, the E series resolvin is selected from RvE1, RvE2, RvE3, RvE4, AT-RvE1, AT-RvE2, and AT-RvE3.

In embodiments, the SPM component (A,B) comprises or consists of a D series resolvin. In embodiments, the D series resolvin is selected from RvD1, RvD2, RvD3, RvD4, RvD5, RvD6, and their aspirin-triggered counterparts.

In embodiments, the compound of Formula IV is a mono or bis resolvin magnesium, calcium, or zinc di-lysinate (M-lys-lys, or M-di-lysinate) compound selected from the group consisting of RvE1 M-lys-lys, RvE2 M-lys-lys, RvE3 M-lys-lys, RvE4 M-lys-lys, AT-RvE1 M-lys-lys, AT-RvE2 M-lys-lys, and AT-RvE3 M-lys-lys.

In embodiments, the compound of Formula IV is a mono or bis resolvin magnesium, calcium, or zinc di-lysinate (M-lys-lys, or M-di-lysinate) compound selected from the group consisting of RvD1 M-lys-lys, RvD2 M-lys-lys, RvD3 M-lys-lys, RvD4 M-lys-lys, RvD5 M-lys-lys, RvD6 M-lys-lys, and their aspirin-triggered counterparts.

In embodiments, the compound of Formula IV is a mono or bis resolvin Mg-di-lysinate compound selected from the group consisting of RvE1 Mg-lys-lys, RvE2 Mg-lys-lys, RvE3 Mg-lys-lys, RvE4 Mg-lys-lys, AT-RvE1 Mg-lys-lys, AT-RvE2 Mg-lys-lys, and AT-RvE3 Mg-lys-lys.

In embodiments, the compound of Formula IV is a mono or bis resolvin Ca-di-lysinate compound selected from the group consisting of RvE1 Ca-lys-lys, RvE2 Ca-lys-lys, RvE3 Ca-lys-lys, RvE4 Ca-lys-lys, AT-RvE1 Ca-lys-lys, AT-RvE2 Ca-lys-lys, and AT-RvE3 Ca-lys-lys.

In embodiments, the compound of Formula IV is a mono or bis resolvin Zn-di-lysinate compound selected from the group consisting of RvE1 Zn-lys-lys, RvE2 Zn-lys-lys, RvE3 Zn-lys-lys, RvE4 Zn-lys-lys, AT-RvE1 Zn-lys-lys, AT-RvE2 Zn-lys-lys, and AT-RvE3 Zn-lys-lys.

Exemplary compounds of Formula IV are provided in Table 4.

### The SPM Component

The compounds represented by Formulas I-IV each contain at least one or two resolvin molecules, which may be referred to herein as the “SPM component” of the compound, and a scaffold portion to which the SPM component is ionically bound. The terms “mono” and “bis” refer to one (mono) or two (bis) SPM molecules in the salt compound.

In the context of the present disclosure, the term “SPM” refers to resolvins and their aspirin-triggered counterparts (e.g., aspirin-triggered resolvins), as described in more detail infra. Examples of particular SPM molecules that may form the SPM component of the compounds described here, as well as their precursor molecules, are given in Tables 1-4 infra. It is understood that the neutral compounds described in these tables may become charged (i.e., deprotonated) if solvated at the appropriate pH.

In embodiments, the SPM component of a compound described here comprises or consists of one or two SPM molecules selected from mediators derived from eicosapentaenoic acid (EPA) (Table 1); mediators derived from docosahexaenoic acid (DHA) (Table 2); and aspirin-triggered mediators (Table 3). In embodiments, the SPM component of a compound described here comprises or consists of two SPM molecules selected from Tables 1-3. In embodiments, the two SPM molecules are the same or different. In embodiments, the two SPM molecules are the same and are selected from an E series resolvin; in embodiments, the E series resolvin is selected from RvE1, RvE2, RvE3, RvE4, AT-RvE1, AT-RvE2, and AT-RvE3. In embodiments, the two SPM molecules are the same and are selected from the group consisting of RvD1, RvD2, RvE1, AT-RvD1, AT-RvD2, and AT-RvE1. In embodiments, the SPM component of a compound described here consists of one or two SPM molecules selected from an E series resolvin; in embodiments, the E series resolvin is selected from RvE1, RvE2, RvE3, RvE4, AT-RvE1, AT-RvE2, and AT-RvE3. In embodiments, the SPM component of a compound described here consists of one or two SPM molecules selected from the group consisting of RvD1, RvD2, RvE1, AT-RvD1, AT-RvD2, and AT-RvE1. In embodiments, the SPM component is selected from an E series resolvin; in embodiments, the E series resolvin is selected from RvE1, RvE2, RvE3, RvE4, AT-RvE1, AT-RvE2, and AT-RvE3. In embodiments, the SPM component is selected from RvE1, AT-RvE1, and RvD1.

In embodiments, the SPM component of a compound or composition described here is selected from a compound set forth in Table 1, Table 2, or Table 3.

TABLE 1
EPA and Resolvins Derived from EPA

| Name | Abbrev. | Formula | Chemical Name |
|---|---|---|---|
| Eicosapentaenoic | EPA | C20H30O2 | 5Z,8Z,11Z,14Z,17Z- |
| acid | | | eicosapentaenoic acid |
| Resolvin E1 | RvE1 | C20H30O5 | 5S,12R,18R-trihydroxy- |
| | | | 6Z,8E,10E,14Z,16E- |
| | | | eicosapentaenoic acid |
| Resolvin E2 | RvE2 | C20H30O4 | 5S,18R-dihydroxy- |
| | | | 6E,8Z,11Z,14Z,16E- |
| | | | eicosapentaenoic acid |
| Resolvin E3 | RvE3 | C20H30O4 | 17R,18R-dihydroxy- |
| | | | 5Z,8Z,11Z,13E,15E- |
| | | | eicosapentaenoic acid |
| Resolvin E4 | RvE₄ | C20H30O4 | 5S,15S-dihydroxy- |
| | | | 6E,8Z,11Z,13E,17Z- |
| | | | eicosapentaenoic acid |

TABLE 2
DHA and Resolvins Derived from DHA

| Name | Abbrev. | Formula | Chemical Name |
|---|---|---|---|
| Docosahexaenoic | DHA | C22H32O2 | 4Z,7Z,10Z,13Z,16Z,19Z- |
| acid | | | docosahexaenoic acid |
| Resolvin D1 | RvD1 | C22H32O5 | 7S,8R,17S-trihydroxy- |
| | | | 4Z,9E,11E,13Z,15E,19Z- |
| | | | docosahexaenoic acid |
| Resolvin D2 | RvD2 | C22H32O5 | 7S,16R,17S-trihydroxy- |
| | | | 4Z,8E,10Z,12E,14E,19Z- |
| | | | docosahexaenoic acid |
| Resolvin D3 | RvD3 | C22H32O5 | 4S,11R,17S-trihydroxy- |
| | | | 5Z,7E,9E,13Z,15E,19Z- |
| | | | docosahexaenoic |
| | | | acid |
| Resolvin D4 | RvD4 | C22H32O5 | 4S,5,17S-trihydroxy- |
| | | | 6E,8E,10Z,13Z,15E,19Z- |
| | | | docosahexaenoic acid |
| Resolvin D5 | RvD5 | C22H32O4 | 7S,17S-dihydroxy- |
| | | | 4Z,8E,10Z,13Z,15E,19Z- |
| | | | docosahexaenoic acid |
| Resolvin D6 | RvD6 | C22H32O4 | 4S,17S-dihydroxy- |
| | | | 5E,7Z,10Z,13Z,15E,19Z- |
| | | | docosahexaenoic acid |

TABLE 3
Aspirin-Triggered Resolvins

| Name | Abbrev. | Formula | Chemical Name |
|---|---|---|---|
| Aspirin-triggered | AT-RvD1 | C22H32O5 | 7S,8R,17R-trihydroxy- |
| Resolvin D1 | | | 4Z,9E,11E,13Z,15E,19Z-docosahexaenoic |
| Aspirin-triggered | AT-RvD2 | C22H32O5 | 7S,16R,17R-trihydroxy- |
| Resolvin D2 | | | 4Z,8E,10Z,12E,14E,19Z-docosahexaenoic |
| | | | acid |
| Aspirin-triggered | AT-RvD3 | C22H32O5 | 4S,11R,17R-trihydroxy- |
| Resolvin D3 | | | 5Z,7E,9E,13Z,15E,19Z-docosahexaenoic |
| | | | acid |
| Aspirin-triggered | AT-RvD4 | C22H32O4 | 4S,5,17R-trihydroxy- |
| Resolvin D4 | | | 6E,8E,10Z,13Z,15E,19Z-docosahexaenoic |
| | | | acid |
| Aspirin-triggered | AT-RvD5 | C22H32O4 | 7S,17R-dihydroxy- |
| Resolvin D5 | | | 4Z,8E,10Z,13Z,15E,19Z-docosahexaenoic |
| | | | acid |
| Aspirin-triggered | AT-RvD6 | C22H32O4 | 4S,17R-dihydroxy- |
| Resolvin D6 | | | 5E,7Z,10Z,13Z,15E,19Z-docosahexaenoic |
| | | | acid |
| Aspirin-triggered | AT-RvE1 | C20H30O5 | 5S,12R,18S-trihydroxy- |
| Resolvin E1 | | | 6Z,8E,10E,14Z,16E-eicosapentaenoic acid |
| Aspirin-triggered | AT-RvE2 | C20H30O4 | 5S,18S-dihydroxy-6E,8Z,11Z,14Z,16E- |
| Resolvin E2 | | | eicosapentaenoic acid |
| Aspirin-triggered | AT-RvE3 | C20H30O4 | 17R,18S-dihydroxy-5Z,8Z,11Z,13E,15E- |
| Resolvin E3 | | | eicosapentaenoic acid |

### Pharmaceutical Compositions

The present disclosure provides pharmaceutical compositions comprising a resolvin, or its salt, and a pharmaceutically acceptable carrier. In embodiments, the salt of the resolvin may be a simple salt, such as a sodium, potassium, or magnesium salt, or a compound of Formula I-IV.

In embodiments, the pharmaceutical composition is formulated as a parenteral dosage form such as a sterile aqueous solution or dispersion suitable for parenteral administration. In embodiments, the parenteral dosage form is selected from an intravenous dosage form, an intra-arterial dosage form, or an intramuscular dosage form. In embodiments, the dosage form is suitable for administration by a subcutaneous route.

In embodiments, the pharmaceutical composition is in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof.

In accordance with any of these embodiments, the dosage form may be in the form of a clear aqueous solution, which may optionally be frozen, or in the form of a lyophilized solid, preferably a sterile lyophilized solid e.g., contained in container, such as a pre-filled syringe, vial or ampule. In some embodiments, the container contains lyophilized composition and is suitable for reconstitution with a specified amount of sterile water or aqueous buffer for administration by a parenteral route, e.g., intravenous, intra-arterial, or an intramuscular, subcutaneous.

In embodiments, the pharmaceutical composition is formulated as an oral or peroral dosage form. In embodiments, the oral formulation is in the form of e.g., a tablet, capsule, powder, solution, suspension, or emulsion.

In embodiments, the pharmaceutical composition is formulated as a sublingual dosage form. In embodiments, the sublingual formulation is in the form of a tablet, film or spray.

In embodiments, the pharmaceutical composition is formulated for administration via inhalation through the nose or mouth. In embodiments, the inhalable dosage form is a liquid formulation, such as an aqueous solution formulation adapted for pulmonary delivery via a nebulizer, including jet, vibrating mesh, and static mesh or orifice nebulizers. In embodiments, the inhalable dosage form is a dry powder for inhalation (DPI). In embodiments, the inhalable dosage form is a propellant-based aerosol formulation suitable for administration using a metered dose inhaler (MDI).

In embodiments, a pharmaceutical composition comprising a resolvin, or its salt, or a compound of any one of Formulas I-IV is in the form of a unit dose of the resolvin, its salt, or the compound. In embodiments, the unit dosage form is a sterile, freeze-dried composition in a suitable container, such as an ampule or vial. The term “freeze-dried” is synonymous with the term “lyophilized” in this context. In embodiments, the unit dose contains from 1 microgram (ug) to 50 milligrams (mg) of the resolvin free acid or salt. In embodiments, the unit dose contains 1, 5, 10, 25, 50, 100, 250, or 500 micrograms of the resolvin free acid or salt. In embodiments, the unit dose contains 1, 5, 10, or 20 milligrams of the resolvin free acid or salt.

The compositions described here may be formulated using one or more suitable excipients or carriers. A suitable excipient or carrier is one suitable for human or animal use. The term “excipient” refers to an additive that serves some purpose in the composition other than a carrier, for example as a stabilizer, solubilizing agent, or suspending agent. Often, a carrier will serve a dual purpose as a simple carrier or diluent and an excipient. Examples of pharmaceutically acceptable excipients may thus include carriers. Non-limiting examples of excipients for use in the compositions of the invention include sterile liquids, water, buffered saline, ethanol, polyols (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, and suitable mixtures thereof.

A suitable excipient or carrier is typically a pharmaceutically acceptable carrier or excipient for use in animals or humans (or both). The term “pharmaceutically acceptable” indicates approval by a regulatory agency of the Federal or a state government or listed in the U.S. Pharmacopeia or other generally recognized pharmacopeia such as the European Pharmacopeia, for use in animals, and more particularly in humans. In the context of the pharmaceutical compositions of the invention, a “carrier” refers to, for example, a solvent, a diluent, or vehicle with which the ionic salt of the invention is formulated for delivery. Examples of pharmaceutically acceptable carriers for use in the compositions of the invention include, without limitation, sterile aqueous and non-aqueous liquids, water, buffered saline, ethanol, polyols (for example, glycerol, propylene glycol, liquid polyethylene glycol and the like), and oils, for liquid dosage forms; or carbohydrates (e.g., glucose, lactose, sucrose or dextran) for solid dosage forms.

The disclosure also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use, one or more syringes, one or more applicators, or a sterile solution suitable for reconstituting a pharmaceutical composition of the present invention.

A representative compound of Formula IV, bis RvE1 magnesium di-lysinate (Compound 1), was tested for efficacy in several preclinical mouse models including the Panc2-H7 model of pancreatic cancer, and the MC38 model of colorectal cancer. These models are well established, highly tumorigenic syngeneic models in which murine cancer cell lines are injected subcutaneously (SC) into the dorsum of C57BL/6J mice. If left untreated, these tumors grow rapidly and lead to death or euthanasia, per protocol. As detailed below in Examples 1-4 and the accompanying figures, bis RvE1 Mg-di-lysinate (Compound 1) was unexpectedly effective as an anti-cancer agent in each of these model systems when administered in a less than daily (LTD) dosing regimen. This was unexpected based on the pharmacokinetics of this compound, which indicated that administration of resolvins in a less than daily (LTD) dosing regimen was unlikely to be effective therapeutically, or at least that it is unlikely to be as effective as daily or more frequent dosing or continuous infusion (see Example 4). Without being bound by any theory, the surprising efficacy of LTD dosing of resolvins in the model systems described here may be the result of durable effects on the inflammatory phenotype of the tumor microenvironment including changes in immune cell phenotype, vascularization, stromal phenotype, and antigen presentation, as well as durable effects on the T-cell compartment such as T-cell priming and increased tumor-infiltrating lymphocytes. In support of this mechanism, RNAseq analysis of pancreatic tumors in mice following treatment with bis RvE1 Mg-di-lysinate shows modulation of T-cells and other immune cells indicative of activation of an adaptive immune response (see Example 5).

### Example 1—Study of Bis RvE1 Mg-Di-Lysinate Regimen with QD and Q3D Subcutaneous Injection in a Preclinical Colorectal Cancer Model

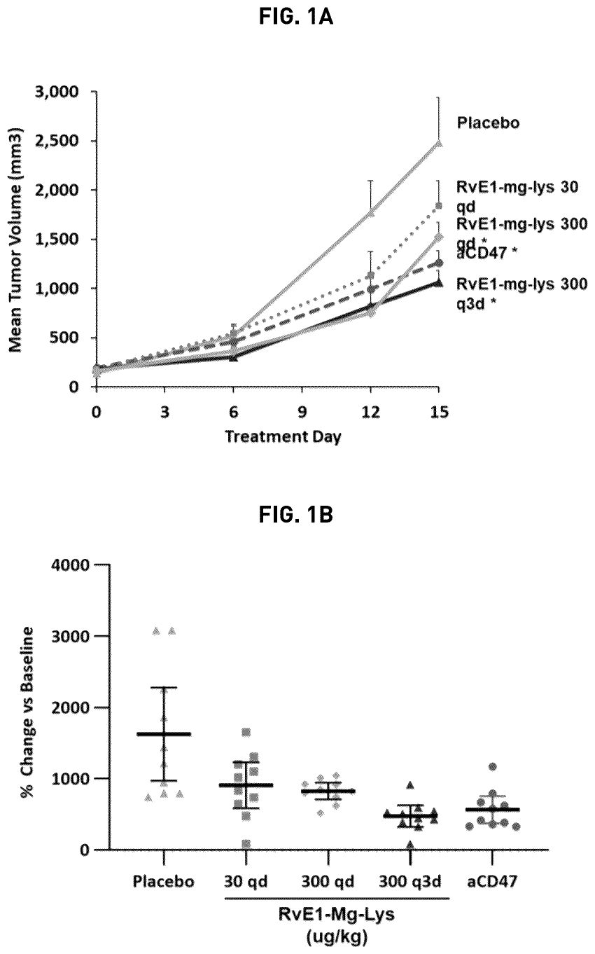

The MC38 model of colorectal cancer was used to investigate whether subcutaneous dosing of bis RvE1 Mg-di-lysinate would be useful in treating cancer and to explore the efficacy of less than daily (LTD) dosing compared to daily dosing. Forty (40) C57BL/6J male mice were injected subcutaneously with 1×10e6 living MC-38 cells. Treatment was initiated when tumors were 150-200 mm\hat{ }3 with bis RvE1 Mg-di-lysinate (30 or 300 ug/g, SC, QD or 300 ug/kg, SC, Q3D), or anti-CD47 (16 mg/kg, IP, Q3D). Mice were sacrificed when the average tumor size of the placebo group reached approximately 2,000 mm\hat{ }3. As shown in FIG. 1A to FIG. 1B, bis RvE1 Mg-di-lysinate was effective as a single agent with both QD and Q3D dosing at 300 ug/kg dose (* P<0.05 vs placebo) with comparable effects to anti-CD47, which independently has been shown to be efficacious in this model and is currently being clinically tested for colorectal cancer. Thus, the study confirmed that subcutaneous dosing with bis RvE1 Mg-di-lysinate is efficacious in an established tumor model on par with a clinical candidate when administered in an LTD dosing regimen. This was unexpected and surprising given the short in vivo half-life of resolvin E1.

### Example 2—Comparison of Daily and Weekly Subcutaneous Administration of Bis RvE1 Mg-Di-Lysinate at Two Doses in a Preclinical Model of Pancreatic Cancer

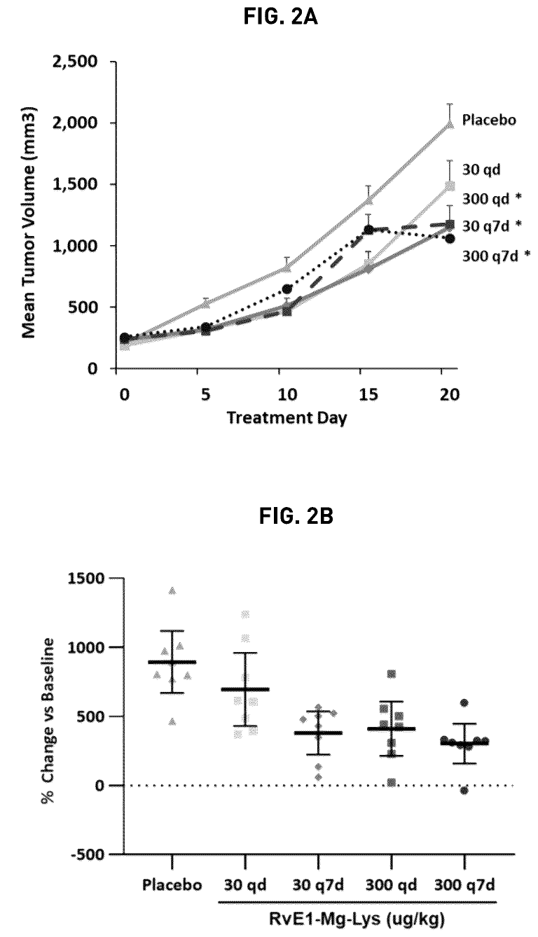

Based on the previous findings in the MC38 colorectal cancer model with less than daily (LTD) dosing, a study in the Panc2-H7 model was conducted to assess the efficacy of LTD dosing of bis RvE1 Mg-di-lysinate in a different tumor model. A total of forty (40) C57BL/6J male mice were injected subcutaneously with 1×10e6 living Panc2-H7 cells. Once tumors reached 160-320 mm\hat{ }3 in size, the mice were randomized (n=8 per group) to treatment with vehicle or SC injection with bis RvE1 Mg-di-lysinate at 30 ug/kg and 300 ug/kg (QD, Q7D). As shown in FIG. 2A to FIG. 2B, bis RvE1 Mg-di-lysinate demonstrated inhibition relative to placebo control at the 30 and 300 ug/kg doses with both QD and Q7D dosing, with the greatest inhibition observed in the 300 ug/kg Q7D group (* P<0.05 vs placebo control). These results were consistent with the unexpected results observed in the MC38 model with LTD dosing, demonstrating that resolvins can be administered as infrequently as once per week and still exhibit potent anti-cancer activity. Based on these results, weekly (Q7D) dosing of bis RvE1 Mg-di-lysinate in combination with other therapies, or alone as maintenance therapy, could offer a substantial improvement in clinical therapy.

### Example 3—RvE1 Pharmacokinetics in Mice

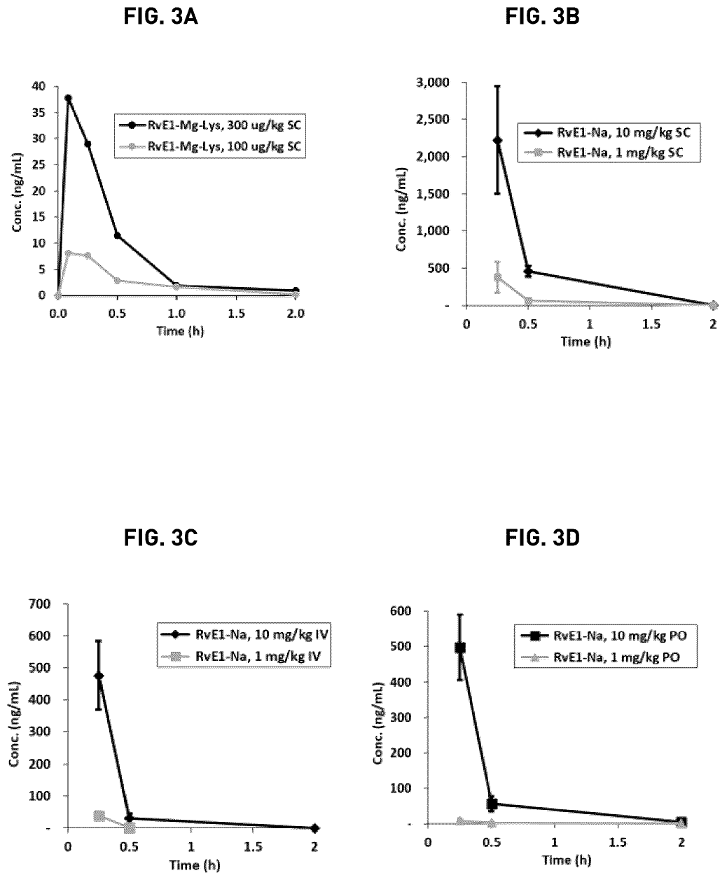

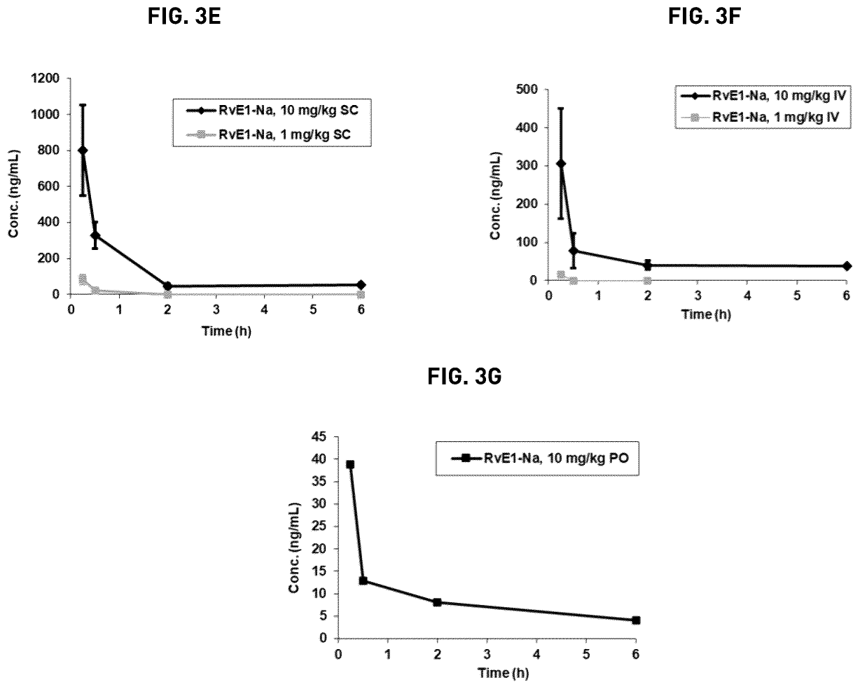

Pharmacokinetic (PK) studies with subcutaneous (FIG. 3A FIG. 3B, FIG. 3E) intravenous (FIG. 3C, FIG. 3F), and oral administration (FIG. 3D, FIG. 3G) of salt forms of RvE1 show rapid depletion of RvE1 from plasma and organ tissues. In the subcutaneous PK study, sixty-nine (69) C57BL/6J mice were administered 100 or 300 ug/kg doses of RvE1-Mg-Lys via subcutaneous injection. Mice were sacrificed and plasma was drawn at the following timepoints: baseline, 5, 15, 30 min, 1, 2, 4 and 8 hours (n=5 mice per timepoint). In the IV and oral PK studies, one-hundred eighty (180) Balb/c mice were administered RvE1-Na at 1 or 10 mg/kg via subcutaneous injection, IV injection, oral gavage (n=6 mice per timepoint). Plasma and lung tissue were assayed at the following timepoints: 0.25, 0.5, 1, 2, 6, 10 and 24 hours. Across multiple routes of administration, salts of RvE1 exhibit a short biological half-life that is consistent with prior studies reporting that resolvins, including RvE1, are rapidly metabolized and cleared from systemic circulation and tissue. Collectively, these PK data suggest that administration of resolvins in a less than daily (LTD) dosing regimen is unlikely to be effective therapeutically, or at least that it is unlikely to be as effective as daily or more frequent dosing or continuous infusion. Without being bound by any theory, the surprising efficacy of LTD dosing of resolvins in the cancer model systems described here may be the result of durable effects on the tumor microenvironment including changes in immune cell phenotype, vascularization, stromal phenotype, and antigen presentation, as well as durable effects on the T-cell compartment such as T-cell priming and increased tumor-infiltrating lymphocytes.

### Example 4—Cell Enrichment Analysis Shows Modulation of Adaptive Immune Response in Pancreatic Cancer

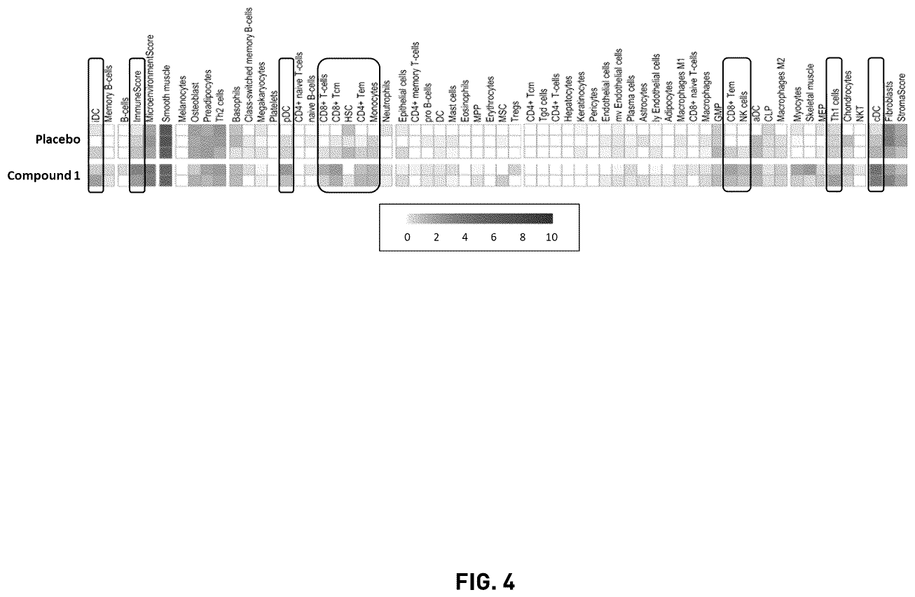

RNAseq analysis was performed on tumor samples from mice with subcutaneous KPC tumors sacrificed on Day 26 following treatment (Q6D) with bis RvE1 Mg-di-lysinate (Compound 1) compared to placebo. FIG. 4 shows a heatmap of gene expression for placebo treated mice and mice treated with Compound 1. Darker color indicates increased gene expression, for various immune cells and other cells, as listed across the top of the heatmap. The boxed cells shown in FIG. 4 indicate an increase in markers of monocytes and DC cell subset (cDCs, pDCs, and iDCs) and markers of NK, CD8+ T cells, Th1 cells, and memory T cells (CD8+ and CD4+) with Compound 1 treatment. These data support a general mechanism for the surprising efficacy of the resolvins in the cancer model systems described above. Namely, these unexpected results may be due to durable effects on the tumor microenvironment including changes in immune cell phenotype and the T-cell compartment such as T-cell priming and increased tumor-infiltrating lymphocytes.

The structures of exemplary compounds of Formulas I and IV are shown below in Table 4. The compounds can be synthesized, for example, as described in U.S. Pat. No. 10,420,843, which is incorporated herein by reference in its entirety. Alternate methods for obtaining the SPM component of a compound described here are described, for example, in Li et al., Beilstein J. Org. Chem. 2013, 9, 2762-2766 and Vik et al., Bioorganic and Med. Chem. Let 2017. In addition, one or more SPMs for use in the SPM component of a compound described here may be available for purchase from a vendor such as Caymen Chemical Co. (Ann Arbor, MI).

TABLE 4
Structures of Representative Compounds of Formulas I and IV

| Name | Structure |
|---|---|
| RvE1- MgLys Compound 1 | 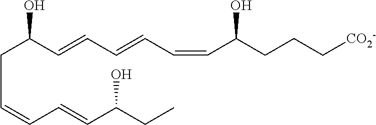 |
| | 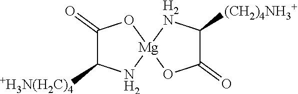 |
| | 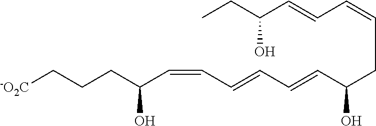 |
| RvE1-CaLys Compound 2 | 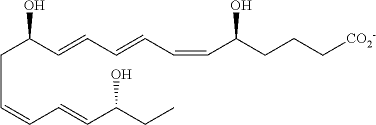 |
| | 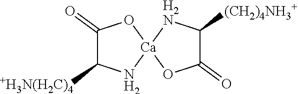 |
| | 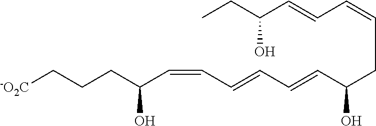 |
| RvE1-ZnLys Compound 3 | 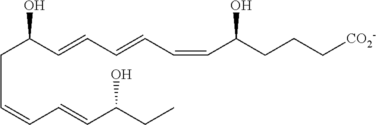 |
| | 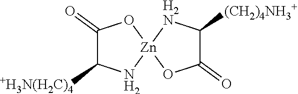 |
| | 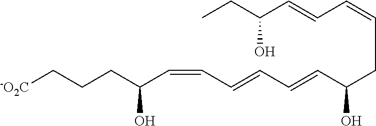 |
| RvE1- LysLys Compound 4 | 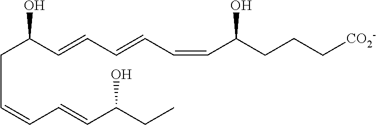 |
| | 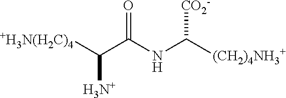 |
| | 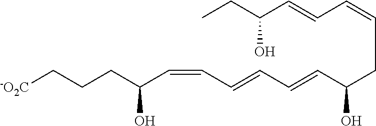 |
| RvE1- LysLys (linear) Compound 5 | 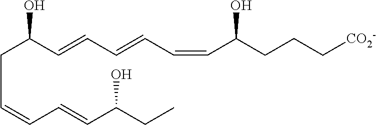 |
| | 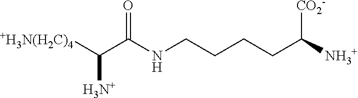 |
| | 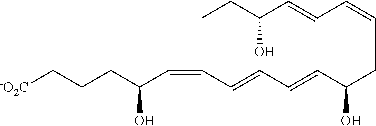 |
| AT(18S)- RvE1- MgLys Compound 6 | 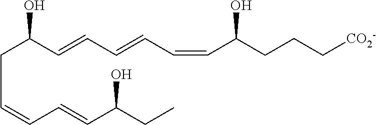 |
| | 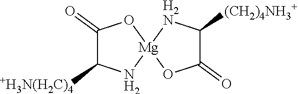 |
| | 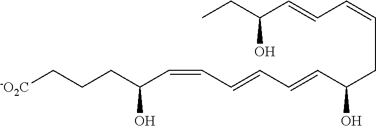 |
| AT(18S)- RvE1- CaLys Compound 7 | 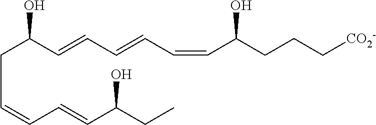 |
| | 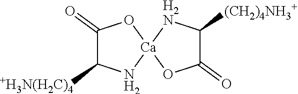 |
| | 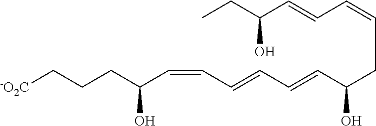 |
| AT(18S)- RvE1- ZnLys Compound 8 | 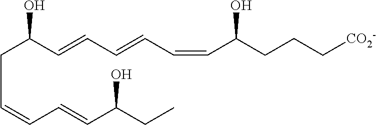 |
| | 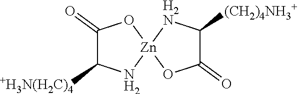 |
| | 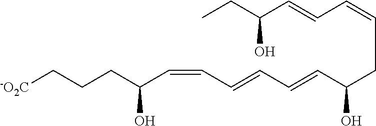 |
| AT(18S)- RvE1- LysLys Compound 9 | 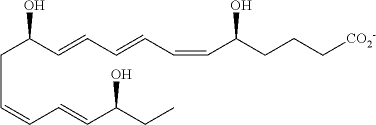 |
| | 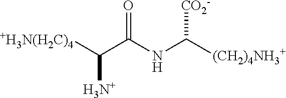 |
| | 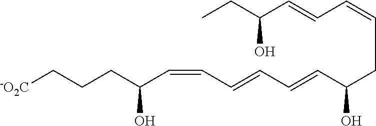 |
| AT(18S)- RvE1- LysLys (linear) Compound 10 | 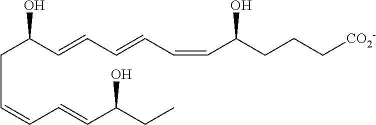 |
| | 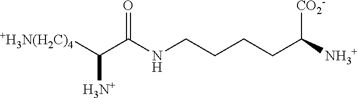 |
| | 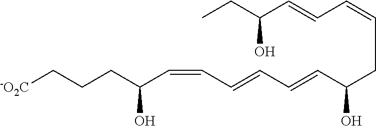 |
| RvD1- MgLys Compound 11 | 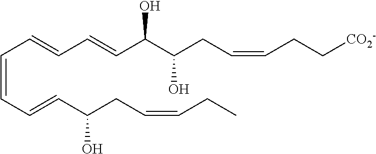 |
| | 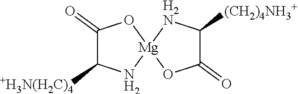 |
| | 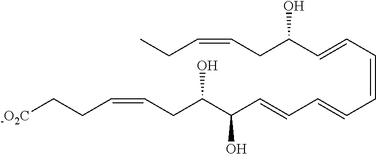 |
| RvD1- CaLys Compound 12 | 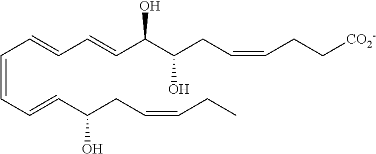 |
| | 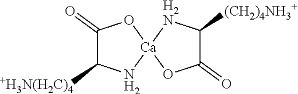 |
| | 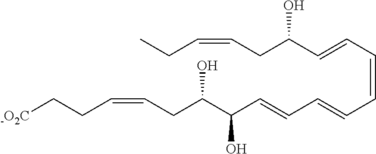 |
| RvD1- ZnLys Compound 13 | 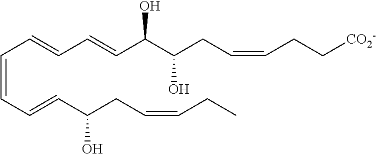 |
| | 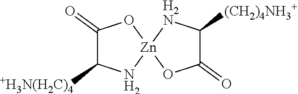 |
| | 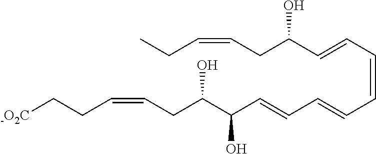 |
| RvD1- LysLys Compound 14 | 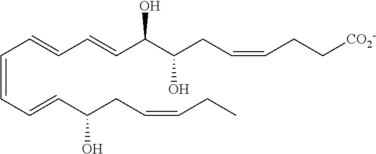 |
| | 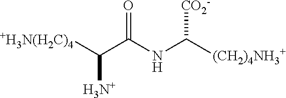 |
| | 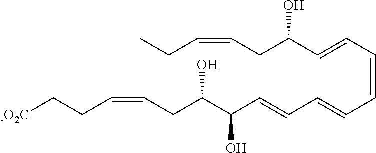 |
| RvD1- LysLys (linear) Compound 15 | 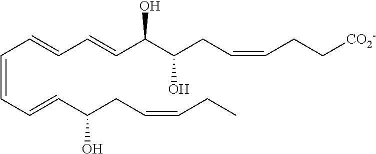 |
| | 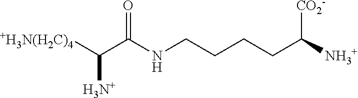 |
| | 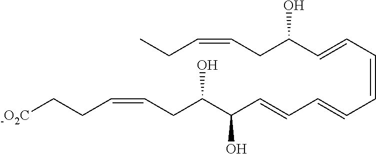 |
| AT(17e)- RvD1- MgLys Compound 16 | 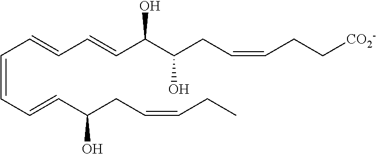 |
| | 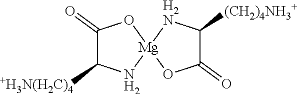 |
| | 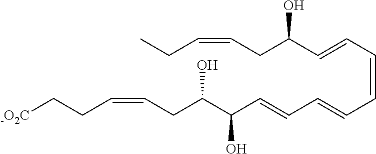 |
| AT(17e)- RvD1- CaLys Compound 17 | 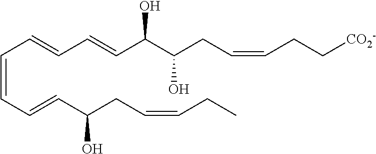 |
| | 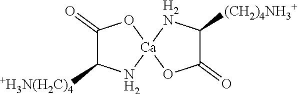 |
| | 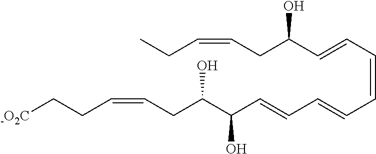 |
| AT(17e)- RvD1- ZnLys Compound 18 | 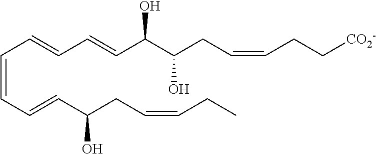 |
| | 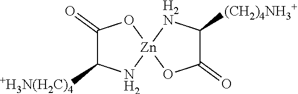 |
| | 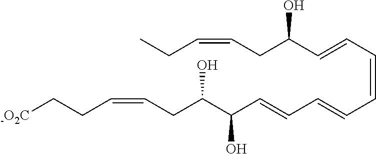 |
| AT(17e)- RvD1- LysLys Compound 19 | 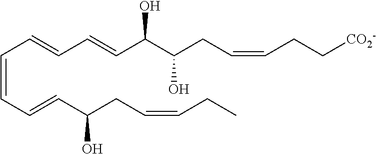 |
| | 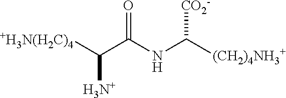 |
| | 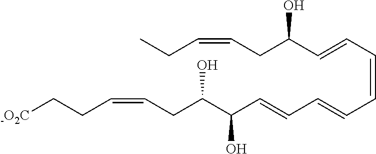 |
| AT(17e)- RvD1- LysLys (linear) Compound 20 | 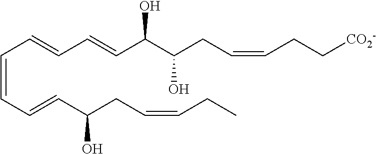 |
| | 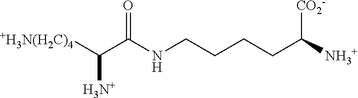 |
| | 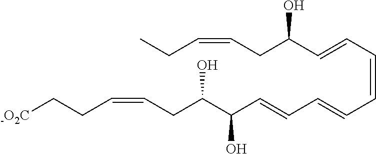 |
| RvD2- MgLys Compound 21 | 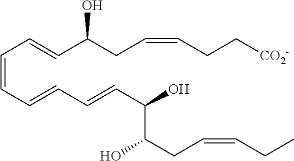 |
| | 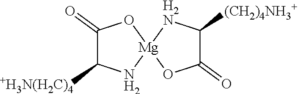 |
| | 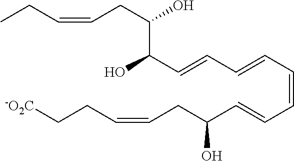 |
| RvD2- CaLys Compound 22 | 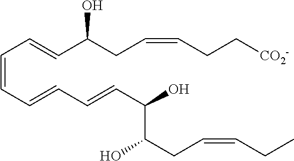 |
| | 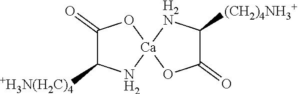 |
| | 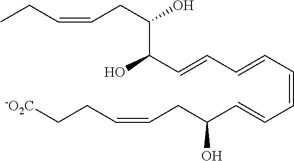 |
| RvD2- ZnLys Compound 23 | 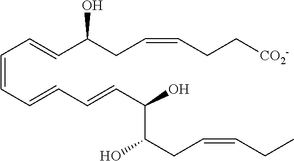 |
| | 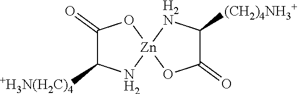 |
| | 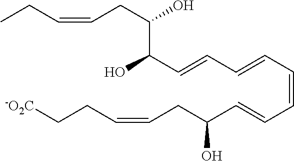 |
| RvD2- LysLys Compound 24 | 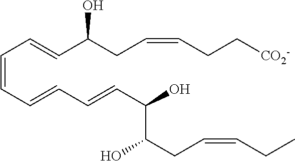 |
| | 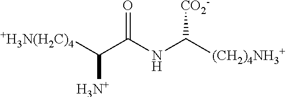 |
| | 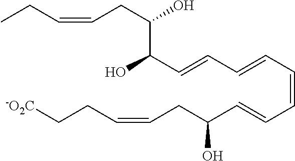 |
| RvD2- LysLys (linear) Compound 25 | 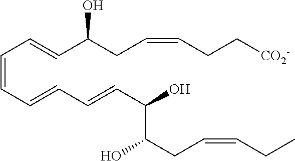 |
| | 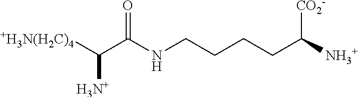 |
| | 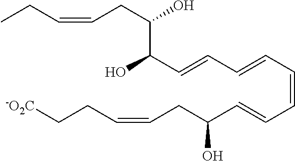 |
| RvE2- MgLys Compound 26 | 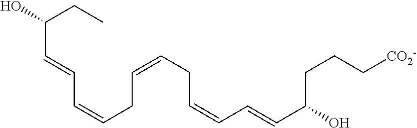 |
| | 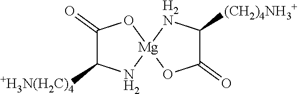 |
| | 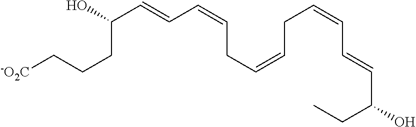 |
| RvE2- CaLys Compound 27 | 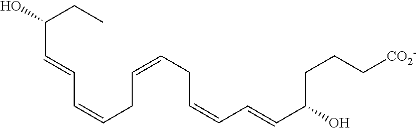 |
| | 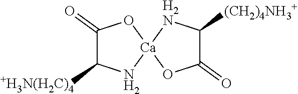 |
| | 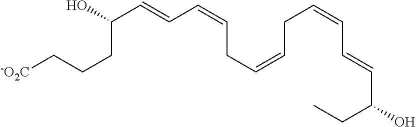 |
| RvE2- ZnLys Compound 28 | 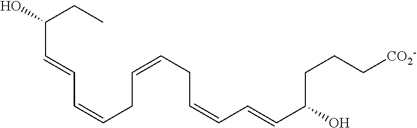 |
| | 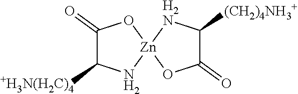 |
| | 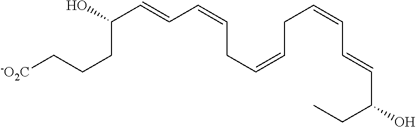 |
| RvE2- LysLys Compound 29 | 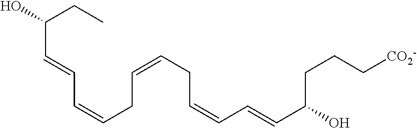 |
| | 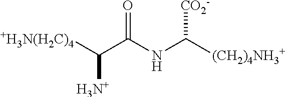 |
| | 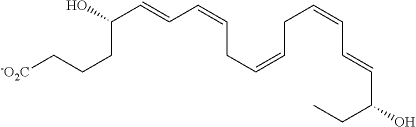 |
| RvE2- LysLys (linear) Compound 30 | 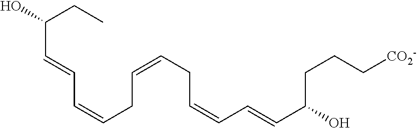 |
| | 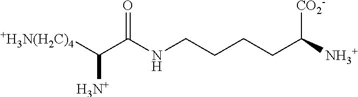 |
| | 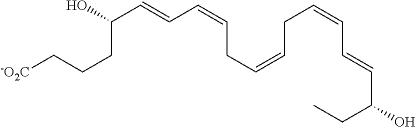 |
| AT-RvE2- MgLys Compound 31 | 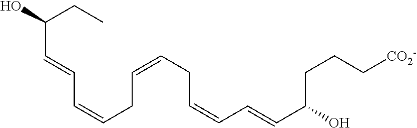 |
| | 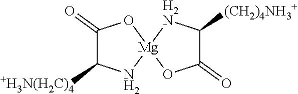 |
| | 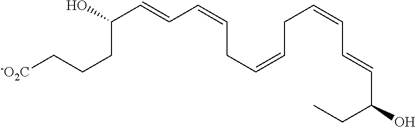 |
| AT-RvE2- CaLys Compound 32 | 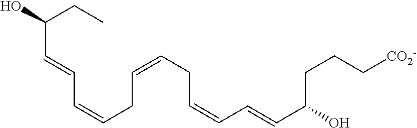 |
| | 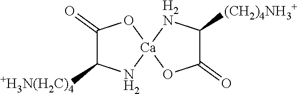 |
| | 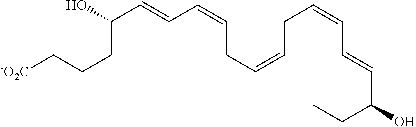 |
| AT-RvE2- ZnLys Compound 33 | 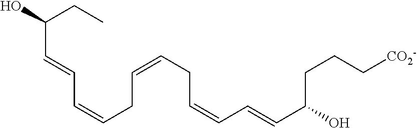 |
| | 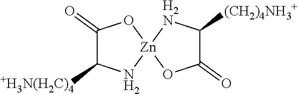 |
| | 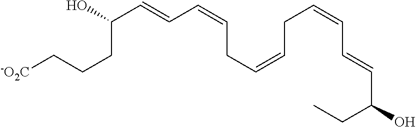 |
| AT-RvE2- LysLys Compound 34 | 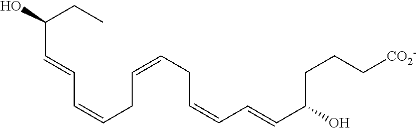 |
| | 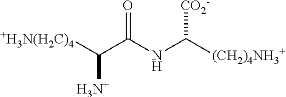 |
| | 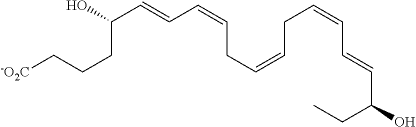 |
| AT-RvE2- LysLys (linear) Compound 35 | 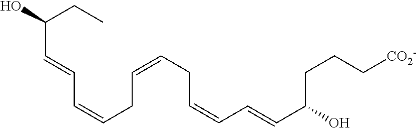 |
| | 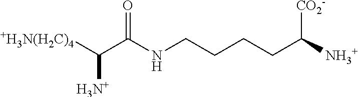 |
| | 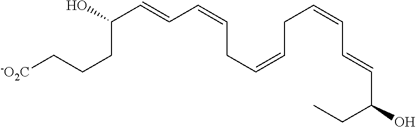 |
| RvE3- MgLys Compound 36 | 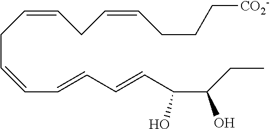 |
| | 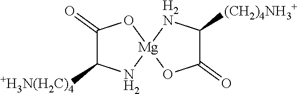 |
| | 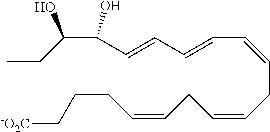 |
| RvE3- CaLys Compound 37 | 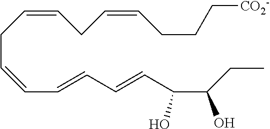 |
| | 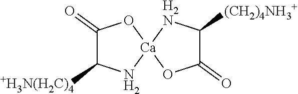 |
| | 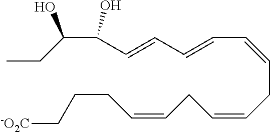 |
| RvE3- ZnLys Compound 38 | 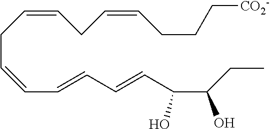 |
| | 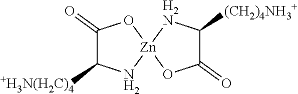 |
| | 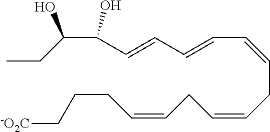 |
| RvE3- LysLys Compound 39 | 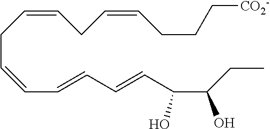 |
| | 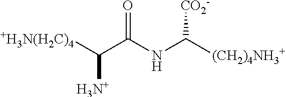 |
| | 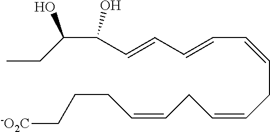 |
| RvE3- LysLys (linear) Compound 40 | 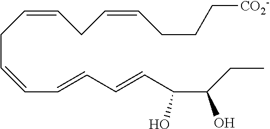 |
| | 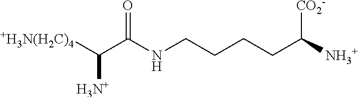 |
| | 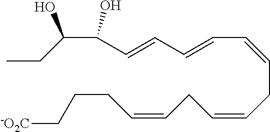 |
| AT-RvE3- MgLys Compound 41 | 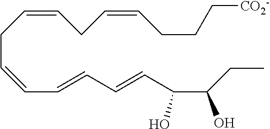 |
| | 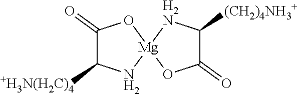 |
| | 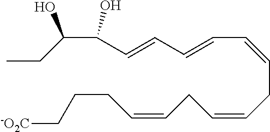 |
| AT-RvE3- CaLys Compound 42 | 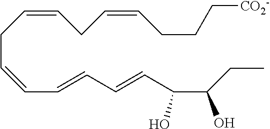 |
| | 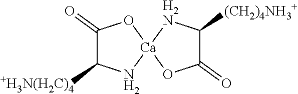 |
| | 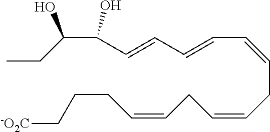 |
| AT-RvE3- ZnLys Compound 43 | 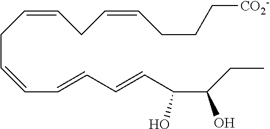 |
| | 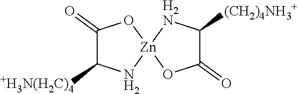 |
| | 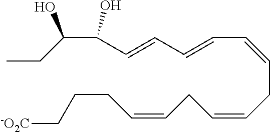 |
| AT-RvE3- LysLys Compound 44 | 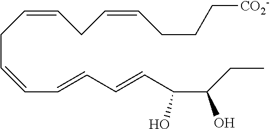 |
| | 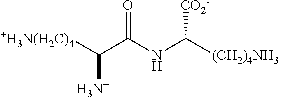 |
| | 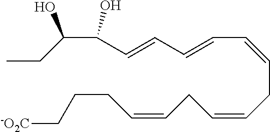 |
| AT-RvE3- LysLys (linear) Compound 45 | 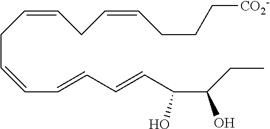 |
| | 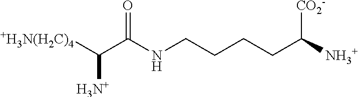 |
| | 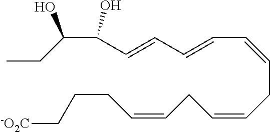 |
| RvE4- MgLys Compound 46 | 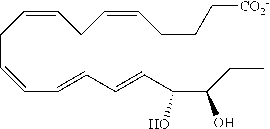 |
| | 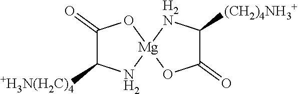 |
| | 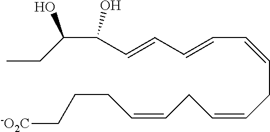 |
| RvE4- CaLys Compound 47 | 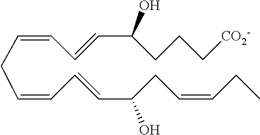 |
| | 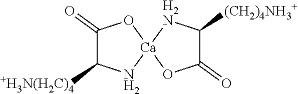 |
| | 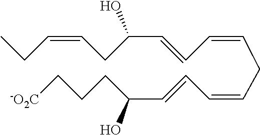 |
| RvE4- ZnLys Compound 48 | 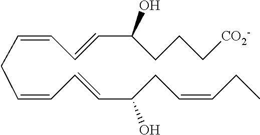 |
| | 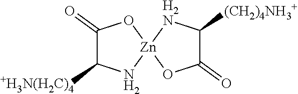 |
| | 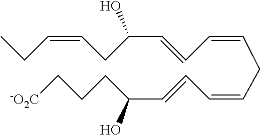 |

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method for treating an immune-mediated inflammatory disease in a subject in need of such treatment, the method comprising administering to the subject a pharmaceutical composition comprising a resolvin, or a salt thereof, in a therapeutic regimen of less than once daily, either alone as monotherapy or as adjuvant or neo-adjuvant therapy, optionally in combination with one or more additional therapeutic agents or therapies.

2. The method of claim 1, wherein the resolvin is a mineral amino acid salt of Formula IV, or an enantiomer, polymorph, solvate, or hydrate thereof: 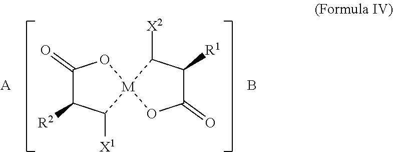
wherein
M is a divalent metal selected from magnesium (Mg²⁺), calcium (Ca²⁺), and zinc (Zn²⁺);
A and B are each independently a resolvin molecule;
A and B may be the same or different;
either A or B, but not both, may be absent;
R¹ and R² are each independently a C₁-C₁₀ alkyl comprising at least one basic function; and
X¹ and X² are each independently H or CO—Z and Z is a peptide comprising 1 to 5 amino acids.

3. The method of claim 1, wherein the resolvin is an E or D series resolvin, optionally wherein the E series resolvin is selected from the group consisting of resolvin E1 (RvE1), resolvin E2 (RvE2), resolvin E3 (RvE3), resolvin E4 (RvE4), aspirin-triggered RvE1 (AT-RvE1), AT-RvE2, and AT-RvE3, and further optionally wherein the D series resolvin selected from the group consisting of resolvin D1 (RvD1), resolvin D2 (RvD2), resolvin D3 (RvD3), resolvin D4 (RvD4), resolvin D5 (RvD5), resolvin D6 (RvD6), aspirin-triggered resolvin D1 (AT-RvD1), AT-RvD2, AT-RvD3, AT-RvD4, AT-RvD5, and AT-RvD6.

4. The method of claim 1, wherein the resolvin is RvE1, RvD1, or RvD2.

5. The method of claim 2, wherein A and B are each independently an E series resolvin selected from the group consisting of resolvin E1 (RvE1), resolvin E2 (RvE2), resolvin E3 (RvE3), resolvin E4 (RvE4), aspirin-triggered RvE1 (AT-RvE1), AT-RvE2, and AT-RvE3.

6. The method of claim 2, wherein A and B are each independently a D series resolvin selected from the group consisting of resolvin D1 (RvD1), resolvin D2 (RvD2), resolvin D3 (RvD3), resolvin D4 (RvD4), resolvin D5 (RvD5), resolvin D6 (RvD6), protectin D1, protectin DX, aspirin-triggered resolvin D1 (AT-RvD1), AT-RvD2, AT-RvD3, AT-RvD4, AT-RvD5, AT-RvD6, and AT protectin D1

7. The method of claim 2, wherein M is selected from magnesium (Mg²⁺) or calcium (Ca²⁺).

8. The method of claim 7, wherein R¹ and R² are each independently —(CH₂)₃—Y¹, and —(CH₂)₄—Y², and Y¹ and Y² are each selected from a positively charged primary amine, a positively charged secondary amine, a positively charged tertiary amine, and a positively charged guanidine,

9. The method of claim 7, wherein X¹ and X² are each H

10. The method of claim 7, wherein R¹ and R² are each —(CH₂)₄—Y² and Y² is —NH₃⁺

11. The method of claim 7, wherein A and B are the same.

12. The method of claim 7, wherein the resolvin is RvE1, RvD1 or RvD2.

13. The method of claim 2, wherein M is magnesium (Mg²⁺), R¹ and R² are each —(CH₂)₄—Y² and Y² is —NH₃⁺, X¹ and X² are each H, and A and B are RvE1, which compound is referred to as bis RvE1 Mg-di-lysinate (Compound 1).

14. The method of claim 2, wherein M is magnesium (Mg²⁺), R¹ and R² are each —(CH₂)₄—Y² and Y² is —NH₃⁺, X¹ and X² are each H, and A and B are RvD1, which compound is referred to as bis RvD1 Mg-di-lysinate.

15. The method of claim 2, wherein M is magnesium (Mg²⁺), R¹ and R² are each —(CH₂)₄—Y² and Y² is —NH₃⁺, X¹ and X² are each H, and A and B are RvD2, which compound is referred to as bis RvD2 Mg-di-lysinate.

16. The method of claim 1, wherein the method comprises administering the pharmaceutical composition comprising a resolvin or its salt less than daily (LTD), optionally every two days (Q2D), every three days (Q3D), every six days (Q6D), every seven days (Q7D), every fourteen days (Q14D), every twenty-one days (Q21D), or every twenty-eight days (Q28D).

17. The method of claim 1, wherein the method comprises administering the pharmaceutical composition comprising a resolvin or its salt in a dosing regimen of once every three days (Q3D), or once every six days (Q6D), or once every seven days (Q7D).

18. The method of claim 1, wherein the immune-mediated inflammatory disease is selected from the group consisting of inflammatory bowel disease (IBD), ulcerative colitis, Crohn's disease, proctitis, pouchitis, Crohn's disease of the pouch, eosinophilic colitis, lymphocytic colitis, collagenous colitis, diversion colitis, chemical colitis, ischemic colitis, eosinophilic esophagitis, Behcet's disease, irritable bowel syndrome, Celiac disease, intestinal mucositis, diverticulitis, and short bowel syndrome, optionally wherein the inflammatory disease or disorder is ulcerative colitis, Crohn's disease, or pouchitis.

19. The method of claim 1, wherein the immune-mediated inflammatory disease is selected from the group consisting of acne, adipose tissue inflammation, allograft rejection, arthritis, bacterial infection, bullous pemphigoid, burn wounds, chelitis, chronic pancreatitis, corneal wound, dermatitis, diabetic wounds, dry eye syndrome, eczema, endometriosis, endotoxin shock, epidermolysis, ankylosing spondylitis, bullosa acquisita, glossitis, heart ischemia, HSV-keratitis, hidradenitis suppurativa, IgA-mediated bullous dermatoses, ischemia reperfusion injury, localized aggressive periodontitis, lupus erythematosus, lyme arthritis, macular edema, oral mucositis, osteoarthritis, periodontitis, peritonitis, pemphigus, postoperative pain, postsurgical cognitive decline, pruritus, psoriasis, psoriatic arthritis, pyoderma gangrenosum, retinopathy, rheumatoid arthritis, scleroderma, Sjogren's syndrome, steroid-induced rosacea, stomatitis, systemic inflammatory response syndrome, temporomandibular joint inflammation, and vascular inflammation.

20. The method of claim 1, wherein the immune-mediated inflammatory disease is selected from the group consisting of bacterial pneumonia, tuberculosis, sepsis, and sepsis-induced cardiomyopathy.

21. The method of claim 1, wherein the immune-mediated inflammatory disease is selected from the group consisting of type 2 diabetes, insulin resistance, hypertriglyceridemia, mixed dyslipidemia, hypercholesterolemia, fatty liver, hypertriglyceridemia, hypercholesterolemia mixed dyslipidemia, nonalcoholic steatohepatitis (NASH), primary biliary syndrome, and primary schlerosing cholangitis.

22. The method of claim 1, wherein the immune-mediated inflammatory disease is selected from the group consisting of Alzheimer's disease, peripheral nerve injury, amyotrophic lateral sclerosis, multiple sclerosis, pain, and fibromyalgia.

23. The method of claim 1, wherein the immune-mediated inflammatory disease is selected from the group consisting of asthma, pulmonary inflammation, bronchiolitis obliterans, bronchopulmonary dysplasia, also referred to as chronic lung disease of infancy, cystic fibrosis, allergic airway response, acute lung injury, acute respiratory distress syndrome, lung injury, idiopathic pulmonary fibrosis, bacterial pneumonia, cigarette smoke-induced lung inflammation, and vascular inflammation.

24. The method of claim 1, wherein the immune-mediated inflammatory disease is a cancer selected from the group consisting of brain cancer, breast cancer, bladder cancer, colorectal cancer, esophageal cancer, gastric cancer, head and neck cancer, liver cancer, lung cancer, melanoma or other skin cancer, ovarian cancer, pancreatic cancer, prostate cancer, renal cell carcinoma, and sarcoma.

25. The method of claim 1, wherein the pharmaceutical composition is administered orally or parenterally, optionally wherein the parenteral administration is subcutaneous, intraperitoneal, intramuscular, or intravenous; optionally wherein the pharmaceutical compositions is administered sublingually or by inhalation.

26. The method of claim 1, wherein the method comprises administering the resolvin, or a salt thereof, in combination with one or more additional therapeutic agents, or one or more additional therapies, such as surgery, radiation therapy, chemotherapy, targeted therapy, biological therapy, non-steroidal anti-inflammatory drugs (NSAIDS), glucocorticoids, disease-modifying antirheumatic drugs (DMARDs), immunosuppressants, aminosalicylates, immunotherapy, hormone therapy, gene therapy, or microbiome therapy.

27. Use of a pharmaceutical composition comprising a resolvin, or a salt thereof, in a method for treating immune-mediated inflammatory disease in a subject, wherein the composition is adapted for administration less than once daily (LTD), optionally wherein the method comprises administering the resolvin, or a salt thereof, in combination with one or more additional therapeutic agents, or one or more additional therapies, such as surgery, radiation therapy, chemotherapy, targeted therapy, biological therapy, non-steroidal anti-inflammatory drugs (NSAIDS), glucocorticoids, disease-modifying antirheumatic drugs (DMARDs), immunosuppressants, aminosalicylates, immunotherapy, hormone therapy, gene therapy, or microbiome therapy.

28. Use claim 27, wherein the composition is adapted for administration in a dosing regimen of once every three days (Q3D), or once every six days (Q6D), or once every seven days (Q7D).
